(19) United States
(12) Patent Application Publication
(10) Pub. No.: US 2014/0221424 A1
(43) Pub. Date: Aug. 7, 2014

(54) PHARMACEUTICAL COMPOSITIONS FOR USE IN THE TREATMENT OF CYSTIC FIBROSIS

(71) Applicant: Vertex Pharmaceuticals Incorporated, Boston, MA (US)

(72) Inventor: Jiuhong Zha, Chicago, IL (US)

(21) Appl. No.: 14/168,928

(22) Filed: Jan. 30, 2014

Related U.S. Application Data
(60) Provisional application No. 61/758,724, filed on Jan. 30, 2013.
(60) Provisional application No. 61/905,522, filed on Nov. 18, 2013.

(51) Int. Cl. A61K 31/47

(52) U.S. Cl. CPC A61K 31/47
USPC 514/312; 546/156

(57) ABSTRACT

The present invention relates to the use of N-[2,4-bis(1,1-dimethylethyl)-5-hydroxyphenyl]-1,4-dihydro-4-oxoquinoline-3-carboxamide and pharmaceutical compositions thereof for the treatment of cystic fibrosis, in patients, including kits and/or products thereof.

### CROSS-REFERENCE TO RELATED APPLICATIONS

This application claims the benefit under 35 U.S.C. §119 of U.S. Provisional Patent Application Ser. No. 61/758,724, filed Jan. 30, 2013, and entitled “Pharmaceutical Compositions for Use in the Treatment of Cystic Fibrosis”, and U.S. Provisional Patent Application Ser. No. 61/905,522, filed Nov. 18, 2013, and entitled “Pharmaceutical Compositions for Use in the Treatment of Cystic Fibrosis”; the entire contents of each of the above applications are incorporated herein by reference.

### FIELD OF THE INVENTION

The present invention relates to the use of N-[2,4-bis(1,1-dimethylethyl)-5-hydroxyphenyl]-1,4-dihydro-4-oxoquinoline-3-carboxamide and pharmaceutical compositions thereof for the treatment of cystic fibrosis, in patients, including kits and/or products thereof.

### BACKGROUND OF THE INVENTION

Cystic fibrosis (CF) is a recessive genetic disease that affects approximately 30,000 children and adults in the United States and approximately 30,000 children and adults in Europe. Despite progress in the treatment of CF, there is no cure.

CF is caused by mutations in the cystic fibrosis transmembrane conductance regulator gene (CFTR) that encodes an epithelial chloride ion channel responsible for aiding in the regulation of salt and water absorption and secretion in various tissues. Small molecule drugs, known as potentiators that increase the probability of CFTR channel opening, represent one potential therapeutic strategy to treat CF. Potentiators of this type are disclosed in WO 2006/002421, which is herein incorporated by reference in its entirety.

### SUMMARY OF THE INVENTION

Provided herein are various aspects and embodiments that relate to the treatment of cystic fibrosis patients.

In one aspect, a method provides a treatment of cystic fibrosis in patients with hepatic impairment.

In another aspect, a method provides a treatment of cystic fibrosis in patients on a regimen including a moderate or strong CYP3A inhibitor.

In a further aspect, a method provides a treatment of cystic fibrosis in patients on a regimen comprising a CYP3A or a P-gp substrate.

In yet another aspect, a method provides a treatment of cystic fibrosis in patients on a certain dietary regimen.

In still a further aspect, a method provides a treatment of cystic fibrosis that includes monitoring the patient's transaminase elevation during treatment.

Additional aspects provide a product that includes a) ivacaftor formulated as KALYDECO™ or bioequivalent drug product thereof; and b) prescribing information for administering KALYDECO™ or a bioequivalent drug product thereof.

### BRIEF DESCRIPTION OF THE DRAWINGS

FIG. 1: Impact of KALYDECO™ on other drugs.

FIG. 2: Impact of other drugs on KALYDECO™.

FIG. 3A: Mean absolute change from baseline in percent predicted FEV₁: Trial 1.

FIG. 3B: Mean absolute change from baseline in percent predicted FEV₁: Trial 2.

### DETAILED DESCRIPTION OF THE INVENTION

### I. Definitions

As used herein, the following definitions shall apply unless otherwise indicated.

The term “CFTR” as used herein means the cystic fibrosis transmembrane conductance regulator protein.

The term “CFTR” as used herein means cystic fibrosis transmembrane conductance regulator gene.

As used herein, the term “active pharmaceutical ingredient” or “API” refers to a biologically active compound. Exemplary APIs include the CF potentiator N-[2,4-bis(1,1-dimethylethyl)-5-hydroxyphenyl]-1,4-dihydro-4-oxoquinoline-3-carboxamide (Ivacaftor).

The term “modulating” as used herein means increasing or decreasing by a measurable amount.

The term “CFTR gating mutation” as used herein means a CFTR mutation that results in the production of a CFTR protein for which the predominant defect is a low channel open probability compared to normal CFTR (Van Goor, F., Hadida S, and Grootenhuis P., “Pharmacological Rescue of Mutant CFTR function for the Treatment of Cystic Fibrosis”, Top. Med. Chem. 208: 3: 91-120). Gating mutations include, but are not limited to, G551D, G178R, S549N, S549R, G551S, G970R, G1244E, S1251N, S1255P, G1349D.

The term “normal CFTR” or “normal CFTR function” as used herein means wild-type like CFTR without any impairment due to environmental factors such as smoking, pollution, or anything that produces inflammation in the lungs.

The term “reduced CFTR” or “reduced CFTR function” as used herein means less than normal CFTR or less than normal CFTR function.

As used herein, the term “amorphous” refers to a solid material having no long range order in the position of its molecules. Amorphous solids are generally supercooled liquids in which the molecules are arranged in a random manner so that there is no well-defined arrangement, e.g., molecular packing, and no long range order. Amorphous solids are generally isotropic, i.e. exhibit similar properties in all directions and do not have definite melting points. For example, an amorphous material is a solid material having no sharp characteristic crystalline peaks) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. See, US 2004/0006237 for a comparison of XRPDs of an amorphous material and crystalline material.

As used herein, the term “substantially amorphous” refers to a solid material having little or no long range order in the position of its molecules. For example, substantially amorphous materials have less than about 15% crystallinity (e.g., less than about 10% crystallinity or less than about 5% crystallinity). It is also noted that the term ‘substantially amorphous’ includes the descriptor, ‘amorphous’, which refers to materials having no (0%) crystallinity.

As used herein, the term “dispersion” refers to a disperse system in which one substance, the dispersed phase, is distributed, in discrete units, throughout a second substance (the continuous phase or vehicle). The size of the dispersed phase can vary considerably (e.g. single molecules, colloidal particles of nanometer dimension, to multiple microns in size). In general, the dispersed phases can be solids, liquids, or gases. In the case of a solid dispersion, the dispersed and continuous phases are both solids. In pharmaceutical applications, a solid dispersion can include: an amorphous drug in an amorphous polymer; an amorphous drug in crystalline polymer; a crystalline drug in an amorphous polymer; or a crystalline drug in crystalline polymer. In this invention, a solid dispersion can include an amorphous drug in an amorphous polymer or an amorphous drug in crystalline polymer. In some embodiments, a solid dispersion includes the polymer constituting the dispersed phase, and the drug constitutes the continuous phase. Or, a solid dispersion includes the drug constituting the dispersed phase, and the polymer constitutes the continuous phase.

As used herein, the term “solid dispersion” generally refers to a solid dispersion of two or more components, usually one or more drugs (e.g., one drug (e.g., Ivacaftor)) and polymer, but possibly containing other components such as surfactants or other pharmaceutical excipients, where the drug(s) (e.g., Ivacaftor) is substantially amorphous (e.g., having about 15% or less (e.g., about 10% or less, or about 5% or less)) of crystalline drug (e.g., N-[2,4-bis(1,1-dimethylethyl)-5-hydroxyphenyl]-1,4-dihydro-4-oxoquinoline-3-carboxamide) or amorphous (i.e., having no crystalline drug), and the physical stability and/or dissolution and/or solubility of the substantially amorphous or amorphous drug is enhanced by the other components. Solid dispersions typically include a compound dispersed in an appropriate carrier medium, such as a solid state carrier. For example, a carrier comprises a polymer (e.g., a water-soluble polymer or a partially water-soluble polymer) and can include optional excipients such as functional excipients (e.g., one or more surfactants) or nonfunctional excipients (e.g., one or more fillers). Another exemplary solid dispersion is a co-precipitate or a co-melt of N-[2,4-bis(1,1-dimethylethyl)-5-hydroxyphenyl]-1,4-dihydro-4-oxoquinoline-3-carboxamide with at least one polymer.

As used herein “crystalline” refers to compounds or compositions where the structural units are arranged in fixed geometric patterns or lattices, so that crystalline solids have rigid long range order. The structural units that constitute the crystal structure can be atoms, molecules, or ions. Crystalline solids show definite melting points.

As used herein the phrase “substantially crystalline”, means a solid material that is arranged in fixed geometric patterns or lattices that have rigid long range order. For example, substantially crystalline materials have more than about 85% crystallinity (e.g., more than about 90% crystallinity or more than about 95% crystallinity). It is also noted that the term ‘substantially crystalline’ includes the descriptor ‘crystalline’, which is defined in the previous paragraph.

As used herein, “crystallinity” refers to the degree of structural order in a solid. For example, Ivacaftor, which is substantially amorphous, has less than about 15% crystallinity, or its solid state structure is less than about 15% crystalline. In another example, Ivacaftor, which is amorphous, has zero (0%) crystallinity.

As used herein, an “excipient” is an inactive ingredient in a pharmaceutical composition. Examples of excipients include fillers or diluents, surfactants, binders, glidants, lubricants, disintegrants, and the like.

As used herein, a “disintegrant” is an excipient that hydrates a pharmaceutical composition and aids in tablet dispersion. Examples of disintegrants include sodium croscarmellose and/or sodium starch glycolate.

As used herein, a “diluent” or “filler” is an excipient that adds bulkiness to a pharmaceutical composition. Examples of fillers include lactose, sorbitol, celluloses, calcium phosphates, starches, sugars (e.g., mannitol, sucrose, or the like) or any combination thereof.

As used herein, a “surfactant” is an excipient that imparts pharmaceutical compositions with enhanced solubility and/or wetability. Examples of surfactants include sodium lauryl sulfate (SLS), sodium stearyl fumarate (SSF), polyoxyethylene 20 sorbitan mono-oleate (e.g., Tween™), or any combination thereof.

As used herein, a “binder” is an excipient that imparts a pharmaceutical composition with enhanced cohesion or tensile strength (e.g., hardness). Examples of binders include dibasic calcium phosphate, sucrose, corn (maize) starch, microcrystalline cellulose, and modified cellulose (e.g., hydroxymethyl cellulose).

As used herein, a “glidant” is an excipient that imparts a pharmaceutical compositions with enhanced flow properties. Examples of glidants include colloidal silica and/or talc.

As used herein, a “colorant” is an excipient that imparts a pharmaceutical composition with a desired color. Examples of colorants include commercially available pigments such as FD&C Blue #1 Aluminum Lake, FD&C Blue #2, other FD&C Blue colors, titanium dioxide, iron oxide, and/or combinations thereof.

As used herein, a “lubricant” is an excipient that is added to pharmaceutical compositions that are pressed into tablets. The lubricant aids in compaction of granules into tablets and ejection of a tablet of a pharmaceutical composition from a die press. Examples of lubricants include magnesium stearate, stearic acid (stearin), hydrogenated oil, sodium stearyl fumarate, or any combination thereof.

As used herein, “drug product” means a finished dosage form, e.g., tablet, capsule, or solution that contains the active drug ingredient, generally, but not necessarily, in association with inactive ingredients.

As used herein, “pharmaceutical equivalents” means drug products in identical dosage forms that contain identical amounts of the identical active drug ingredient, i.e., the same salt or ester of the same therapeutic moiety, or, in the case of modified release dosage forms that require a reservoir or overage or such forms as prefilled syringes where residual volume may vary, that deliver identical amounts of the active drug ingredient over the identical dosing period; do not necessarily contain the same inactive ingredients; and meet the identical compendia or other applicable standard of identity, strength, quality, and purity, including potency and, where applicable, content uniformity, disintegration times, and/or dissolution rates.

As used herein, “pharmaceutical alternatives” means drug products that contain the identical therapeutic moiety, or its precursor, but not necessarily in the same amount or dosage form or as the same salt or ester. Each such drug product individually meets either the identical or its own respective compendia or other applicable standard of identity, strength, quality, and purity, including potency and, where applicable, content uniformity, disintegration times and/or dissolution rates.

As used herein, “bioequivalent” means a drug product showing the absence of a significant difference in the rate and extent to which the active ingredient or active moiety in a pharmaceutical equivalent to the drug product becomes available at the site of drug action when administered at the same molar dose under similar conditions in an appropriately designed study, wherein “significant difference” means that the 90% Confidence Intervals (CI) of the test drug product must fit between 80%-125% of the reference drug product (see Online Training Seminar: “The FDA Process for Approving Generic Drugs”; www.fda.gov/Training/For HealthProfessionals/ucm090320.htm).

The Food and Drug Administration (FDA) has issued guidelines regarding bioequivalent drug products including specific recommendations on the tolerable variation of inactive ingredients in a drug product that would likely render it a pharmaceutically equivalent form. See, for example, the FDA's Guidance for Industry: Submission of Summary Bioequivalence Data for ANDAs from May 2011, the entire contents of which are incorporated herein. For instance, formulations with different amounts of excipients are considered to be the same drug product formulation if (a) for an individual excipient, the difference in weight between the formulations being compared is less than or equal to the percentage shown in Table 1 below, and (b) the cumulative total of all excipient weight differences is less than or equal to 10 percent.

TABLE 1
Immediate Release Formulations-
Differences in Excipient Weights

| | Difference (≦) |
|---|---|
| | in Excipient Weights |
| Excipient | Between Two Formulations* |
| Filler | 10 |
| Disintegrant | 6 |
| Starch | 2 |
| Other | |
| Binder | 3 |
| Lubricant | 0.5 |
| Calcium or Magnesium | 2 |
| Stearate | |
| Other | |
| Glidant | 2 |
| Talc | 0.2 |
| Other | |
| Film Coat | 2 |

*Percentage of difference between the formulation proposed for marketing and another experimental formulation.

As used herein, the term “mild hepatic impairment” means a patient who is assessed as Child-Pugh, class A (score=5-6); the term “moderate hepatic impairment” means a patient who is assessed as Child-Pugh, class B (score=7-9); and “severe hepatic impairment” means a patient who is assessed as Child-Pugh, class C (score=10-15);

As used herein, the term “CYP3A inhibitor” refers to any chemical entity that impedes the normal function of the Cytochrome P450 3A (CYP3A) subfamily of genes and proteins. The CYP3A inhibitor can impede the action of the CYP3A gene or the CYP3A protein/enzyme. A “strong CYP3A inhibitor” is an inhibitor that increases the AUC of a substrate for CYP3A by equal or more than 5-fold or decreases CL (clearance) by more than 80%. A “moderate CYP3A inhibitor” is an inhibitor that increases the AUC of a sensitive substrate for CYP3A by less than 5-fold, but equal to or more than 2-fold, or decreases CL by 50-80%. A “weak CYP3A inhibitor” is an inhibitor that increases the AUC of a sensitive substrate for CYP3A by less than 2-fold but equal to or more than 5-fold, or decreases CL by more than 20-50%. Examples of CYP3A inhibitors include, but not limited to, ketoconazole, itraconazole, posaconazole, voriconazole, telithromycin, clarithmycin, fluconazole, and erthromycin. Additionally, information regarding CYP3A inhibitors, including strong, moderate, and weak inhibitors, can be found on the Food and Drug Administration's (FDA) website, the relevant portions of which are incorporated herein.

As used herein, the term “strong CYP3A inducer” means an inducer which decreases the AUC of a substrate for CYP3A by equal or more than 80%. The term “moderate CYP3A inducer” means an inducer which decreases the AUC of a substrate for CYP3A by 50-80%. The term “weak CYP3A inducer” means an inducer which decreases the AUC of a substrate for CYP3A by 20-50%. Examples of CYP3A inducers include, but not limited to, rifampin, rifabutin, phenobarbital, carbamazepine, phenyloin, and St. John's Wort. Additionally, information regarding CYP3A inducers, including strong, moderate, and weak inducers, can be found on the Food and Drug Administration's (FDA) website, the relevant portions of which are incorporated herein.

As used herein, the term “transaminase elevation” means that the levels of transaminases (for example Aspartate Transaminase (AST) and/or Alanine Transaminase (ALT)) in a patient are higher than normal. Methods of measuring transaminase levels in a patient are known to those having skill in the art, for example measuring ALT and AST in relation to the Upper Limit of Normal (ULN) transaminase level.

As used herein, the term “P-gp substrate” or “CYP3A substrate” means any chemical entity which binds or can form a complex with the CYP3A subfamily of proteins or permeability glycoprotein (P-gp). Examples of P-gp substrates or CYP3A substrates include, but not limited to, medicinal drugs that bind to the CYP3A subfamily of proteins or P-gp, such as midazolam, alprazolam, diazepam, triaolam, digoxin, cyclosporine, and tacrolimus. As used herein, “sensitive substrates” of CYP3A refers to drugs whose plasma AUC values have been shown to increase 5-fold or higher when co-administered with a known CYP3A inhibitor. Additionally, information regarding CYP substrates, including sensitive substrates, can be found on the Food and Drug Administration's (FDA) website, the relevant portions of which are incorporated herein.

For further information regarding inhibitors, inducers and substrates of CYP enzymes, as well as further examples thereof, see: www.fda.gov/drugs/developmentapprovalprocess/developmentresources/druginteractionslabeling/ucm093664.htm#classInhibit. The entire contents of which, including links therein, is incorporated herein by reference.

As used herein, the term “Ivacaftor” refers to the compound N-[2,4-bis(1,1-dimethylethyl)-5-hydroxyphenyl]-1,4-dihydro-4-oxoquinoline-3-carboxamide, which has the structure

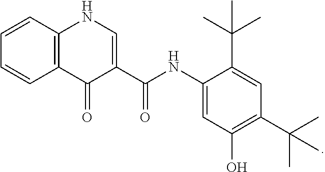 [SMILES: CC(C)(C)c1cc(C(C)(C)C)c(NC(=O)c2c[nH]c3ccccc3c2=O)cc1O]

As used herein, the phrase “Ivacaftor formulated as KALYDECO” refers to a pharmaceutical composition comprising about 34.1 wt % of a solid dispersion by weight of the composition, wherein the dispersion comprises about 80 wt % of substantially amorphous Ivacaftor by weight of the dispersion, about 19.5 wt % of HPMCAS by weight of the dispersion, and about 0.5 wt % SLS by weight of the dispersion; about 30.5 wt % of microcrystalline cellulose by weight of the composition; about 30.4 wt % of lactose by weight of the composition; about 3 wt % of sodium croscarmellose by weight of the composition; about 0.5 wt % of SLS by weight of the composition; about 0.5 wt % of colloidal silicon dioxide by weight of the composition; and about 1 wt % of magnesium stearate by weight of the composition.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools, probes in biological assays or as therapeutic agents.

Examples of suitable solvents are, but not limited to, water, methanol, dichloromethane (DCM), acetonitrile, dimethylformamide (DMF), ethyl acetate (EtOAc), isopropyl alcohol (IPA), isopropyl acetate (IPAc), tetrahydrofuran (THF), methyl ethyl ketone (MEK), t-butanol and N-methylpyrrolidone (NMP).

### II. Embodiments

In some aspects, the method for treating or lessening the severity of cystic fibrosis in a patient, wherein the patient has moderate hepatic impairment, includes administering 150 mg of ivacaftor once daily, wherein the ivacaftor is formulated as KALYDECO™ or a bioequivalent drug product thereof.

In other aspects the method for treating or lessening the severity of cystic fibrosis in a patient, wherein the patient has severe hepatic impairment, includes administering 150 mg of ivacaftor once daily or less frequently, wherein the ivacaftor is formulated as KALYDECO™ or a bioequivalent drug product thereof.

A further aspect includes a method for treating or lessening the severity of cystic fibrosis in a patient, wherein the patient is on a regimen comprising a strong or a moderate CYP3A inhibitor, the method includes administering ivacaftor at a dosage and/or frequency less than the dosage and/or frequency prescribed for patients who are not on the regimen comprising the strong or the moderate CYP3A inhibitor, wherein the ivacaftor is formulated as KALYDECO™ or a bioequivalent drug product thereof. Embodiments of this aspect include one or more of the following features. The patient is a on a regimen comprising a strong CYP3A inhibitor. The method of administering ivacaftor includes administering 150 mg of ivacaftor formulated as KALYDECO™ or a bioequivalent drug product thereof twice-a-week. The strong CYP3A inhibitor is selected from ketoconazole, itraconazole, posaconazole, voriconazole, telithromycin, and clarithmycin. The patient is a on a regimen including a moderate CYP3A inhibitor. The method of administering ivacaftor includes administering 150 mg of ivacaftor formulated as KALYDECO™ or a bioequivalent drug product thereof once daily. The moderate CYP3A inhibitor is fluconazole or erthromycin. The patient limits or refrains from ingesting food comprising grapefruit or Seville oranges. The patient refrains from ingesting food comprising grapefruit or Seville oranges. Ivacaftor formulated as KALYDECO™ or a bioequivalent drug product thereof is administered with fat-containing food. The fat-containing food is selected from eggs, butter, peanut butter, and cheese pizza.

In still another aspect, the method for treating or lessening the severity of cystic fibrosis in a patient, includes administering an effective amount of ivacaftor, wherein the patient is not on a regimen comprising a strong CYP3A inducer. Embodiments of this aspect include one or more of the following features. 150 mg of ivacaftor formulated as KALYDECO™ or a bioequivalent drug product thereof is administered every 12 hours. The patient is not on a regimen comprising rifampin, rifabutin, phenobarbital, carbamazepine, phenyloin, or St. John's Wort. The patient limits or refrains from ingesting food comprising grapefruit or Seville oranges. The patient refrains from ingesting food comprising grapefruit or Seville oranges. Ivacaftor formulated as KALYDECO™ or a bioequivalent drug product thereof is administered with fat-containing food. The fat-containing food is selected from eggs, butter, peanut butter, and cheese pizza.

In yet a further aspect, the method for treating or lessening the severity of cystic fibrosis in a patient, includes administering 150 mg of ivacaftor every 12 hours, wherein the ivacaftor is formulated as KALYDECO™ or a bioequivalent drug product thereof and the patient limits or refrains from ingesting food comprising grapefruit or Seville oranges. In another aspect, the method for treating or lessening the severity of cystic fibrosis in a patient, includes administering 150 mg of ivacaftor every 12 hours, wherein the ivacaftor is formulated as KALYDECO™ or a bioequivalent drug product thereof and the patient refrains from ingesting food comprising grapefruit or Seville oranges. The fat-containing food is selected from eggs, butter, peanut butter, and cheese pizza.

Another aspect provides a method for treating or lessening the severity of cystic fibrosis in a patient including administering an effective amount of ivacaftor with fat-containing food. Embodiments of this aspect include one or more of the following features. The fat-containing food is selected from eggs, butter, peanut butter, and cheese pizza. Ivacaftor is formulated as KALYDECO™ or a bioequivalent drug product thereof 150 mg of ivacaftor formulated as KALYDECO™ or a bioequivalent drug product thereof is administered every 12 hours.

In yet a further aspect, the method for treating or lessening the severity of cystic fibrosis in a patient includes a) administering an effective amount of ivacaftor; b) assessing the patient for transaminase elevation during treatment with ivacaftor; and c) adjusting the effective amount of ivacaftor administered to the patient. Assessing the patient for transaminase elevation during treatment with ivacaftor includes measuring ALT and AST levels and comparing to the Upper Limit of Normal (ULN) transaminase level. Embodiments of this aspect include one or more of the following features. The transaminase levels in a patient prior to initiating treatment with ivacaftor. The patient's transaminase levels are assessed every three months. The patient's transaminase levels are assessed every three months during the first year of treatment with ivacaftor and annually thereafter. The process of assessing the transaminase elevation includes measuring ALT and AST levels. The method further includes interrupting dosing in patients who exhibit elevated ALT and AST level that are greater than five times the upper limit of normal. The effective amount of ivacaftor in step (a) is 150 mg.

Still another aspect provides a method for treating or lessening the severity of cystic fibrosis in a patient, wherein the patient is on a regimen comprising a CYP3A or a P-gp substrate. The method includes administering 150 mg of ivacaftor every 12 hours, wherein the ivacaftor is formulated as KALYDECO™ or a bioequivalent drug product thereof; and b) monitoring side effects related to the regimen comprising the CYP3A or the P-gp substrate. Embodiments of this aspect include one or more of the following features. The regimen including the CYP3A or the P-gp substrate includes midazolam, alprazolam, diazepam, triaolam, digoxin, cyclosporine, or tacrolimus.

In any of the foregoing aspects and embodiments, the patient possesses a CFTR gating mutation in the cystic fibrosis transmembrane conductance regulator gene. In some embodiments the CFTR gating mutation is selected from G551D, G178R, S549N, S549R, G551S, G970R, G1244E, S1251N, S1255P, G1349D. In some embodiments, the CFTR gating mutation is a G551D mutation, and the patient may have the CFTR mutation in one or both alleles. In other embodiments, the CFTR gating mutation is a G178R mutation, and the patient may have the CFTR mutation in one or both alleles. In some embodiments, the CFTR gating mutation is a S549N mutation, and the patient may have the CFTR mutation in one or both alleles. In other embodiments, the CFTR gating mutation is a S549R mutation, and the patient may have the CFTR mutation in one or both alleles. In some embodiments, the CFTR gating mutation is a G551S mutation, and the patient may have the CFTR mutation in one or both alleles. In other embodiments, the CFTR gating mutation is a G970R mutation, and the patient may have the CFTR mutation in one or both alleles. In some embodiments, the CFTR gating mutation is a G1244E mutation, and the patient may have the CFTR mutation in one or both alleles. In other embodiments, the CFTR gating mutation is a S1251N mutation, and the patient may have the CFTR mutation in one or both alleles. In some embodiments, the CFTR gating mutation is a S1255P mutation, and the patient may have the CFTR mutation in one or both alleles. In other embodiments, the CFTR gating mutation is a G1349D mutation, and the patient may have the CFTR mutation in one or both alleles. In any of the foregoing aspects and embodiments, the patient is homozygous for a particular CFTR mutation if the patient has that particular CFTR mutation in both alleles. In any of the foregoing aspects and embodiments, the patient is heterozygous for a particular CFTR mutation if the patient has that particular CFTR mutation only in one allele.

Yet another aspect provides a product that includes a) ivacaftor formulated as KALYDECO™ or bioequivalent drug product thereof; and b) prescribing information for administering KALYDECO™ or a bioequivalent drug product thereof. The prescribing information includes the following: i) dosage and administration information for adults and pediatric patients 6 years and older instructing the administration of one 150 mg tablet of KALYDECO™ or a bioequivalent drug product thereof taken orally every 12 hours with fat-containing food; ii) dosage and administration information to reduce the dose of KALYDECO™ or a bioequivalent drug product thereof in patients with moderate or severe hepatic impairment; and iii) dosage and administration information to reduce the dose of KALYDECO™ or a bioequivalent drug product thereof when co-administered with drugs that are moderate or strong CYP3A inhibitors. Embodiments of this aspect include one or more of the following features. The prescribing information describes fat-containing food as selected from eggs, butter, peanut butter, and cheese pizza. The prescribing information recommends a reduced dose of 150 mg of KALYDECO™ or a bioequivalent drug product thereof once daily in patients with moderate hepatic impairment. The prescribing information recommends a reduced dose of 150 mg of KALYDECO™ or a bioequivalent drug product thereof once daily or less frequently in patients with severe hepatic impairment. The prescribing information recommends reducing the dose of KALYDECO™ or a bioequivalent drug product thereof to 150 mg twice-a-week when co-administered with strong CYP3A inhibitors. The prescribing information describes strong CYP3A inhibitors as selected from ketoconazole, itraconazole, posaconazole, voriconazole, telithromycin, and clarithmycin. The prescribing information recommends reducing the dose of KALYDECO™ or a bioequivalent drug product thereof to 150 mg once daily when co-administered with moderate CYP3A inhibitors. The prescribing information describes moderate CYP3A inhibitors as fluconazole or erthromycin.

In another aspect the product includes a) ivacaftor formulated as KALYDECO™ or bioequivalent drug product thereof; and b) prescribing information for administering KALYDECO™ or a bioequivalent drug product thereof. The prescribing information includes the following: i) drug interaction information to reduce the dose of KALYDECO™ or a bioequivalent drug product thereof to 150 mg of ivacaftor twice-a-week when co-administered with a strong CYP3A inhibitors; ii) drug interaction information to reduce the dose of KALYDECO™ or a bioequivalent drug product thereof to 150 mg of ivacaftor once daily when co-administered with a moderate CYP3A inhibitors; and iii) drug interaction information to avoid food containing grapefruit or Seville oranges.

A further aspect provides a product including a) ivacaftor formulated as KALYDECO™ or bioequivalent drug product thereof; and b) prescribing information for administering KALYDECO™ or a bioequivalent drug product thereof. The prescribing information includes the following i) warnings and precautions regarding elevated transaminases ALT or AST, wherein the prescribing information advises that transaminases ALT and AST should be assessed prior to initiating KALYDECO™ or a bioequivalent drug product thereof, every 3 months during the first year of treatment of KALYDECO™ or a bioequivalent drug product thereof, and annually thereafter; ii) warnings and precautions regarding elevated transaminases ALT or AST, wherein the prescribing information advises that dosing of KALYDECO™ or a bioequivalent drug product thereof should be interrupted in patients with ALT or AST of greater than 5 times the upper limit of normal; and iii) warnings and precautions regarding CYP3A inducers, wherein the prescribing information advises that concomitant use of KALYDECO™ or a bioequivalent drug product thereof with strong CYP3A inducers substantially decreases exposure of ivacaftor which may diminish effectiveness, and co-administration is not recommended. In embodiments of this aspect, the prescribing information describes CYP3A inducers as selected from rifampin, rifabutin, phenobarbital, carbamazepine, phenyloin, and St. John's Wort.

In a further aspect, the product includes a) ivacaftor formulated as KALYDECO™ or bioequivalent drug product thereof; and b) prescribing information for administering KALYDECO™ or a bioequivalent drug product thereof. The package insert includes: i) information regarding the potential for ivacaftor to affect other drugs including CYP3A and P-gp substrates, wherein the prescribing information advises caution when co-administering KALYDECO™ or bioequivalent drug product thereof with CYP3A and/or P-gp substrates. In embodiments of this aspect, the prescribing information describes CYP3A and/or P-gp substrates as selected from midazolam, alprazolam, diazepam, triaolam, digoxin, cyclosporine, and tacrolimus.

Yet another aspect provides a product that includes a) ivacaftor formulated as KALYDECO™ or bioequivalent drug product thereof; and b) prescribing information for administering KALYDECO™ or a bioequivalent drug product thereof. The prescribing information includes the following: i) dosage and administration information for adults and pediatric patients 6 years and older instructing the administration of one 150 mg tablet of KALYDECO™ or a bioequivalent drug product thereof taken orally every 12 hours with fat-containing food; ii) dosage and administration information to reduce the dose of KALYDECO™ or a bioequivalent drug product thereof in patients with moderate or severe hepatic impairment; iii) dosage and administration information to reduce the dose of KALYDECO™ or a bioequivalent drug product thereof when co-administered with drugs that are moderate or strong CYP3A inhibitors; iv) drug interaction information to reduce the dose of KALYDECO™ or a bioequivalent drug product thereof to 150 mg of ivacaftor twice-a-week when co-administered with a strong CYP3A inhibitors; v) drug interaction information to reduce the dose of KALYDECO™ or a bioequivalent drug product thereof to 150 mg of ivacaftor once daily when co-administered with a moderate CYP3A inhibitors; vi) drug interaction information to avoid food containing grapefruit or Seville oranges; vii) warnings and precautions regarding elevated transaminases ALT or AST, wherein the prescribing information advises that transaminases ALT and AST should be assessed prior to initiating KALYDECO™ or a bioequivalent drug product thereof, every 3 months during the first year of treatment of KALYDECO™ or a bioequivalent drug product thereof, and annually thereafter; viii) warnings and precautions regarding elevated transaminases ALT or AST, wherein the prescribing information advises that dosing of KALYDECO™ or a bioequivalent drug product thereof should be interrupted in patients with ALT or AST of greater than 5 times the upper limit of normal; and ix) warnings and precautions regarding CYP3A inducers, wherein the prescribing information advises that concomitant use of KALYDECO™ or a bioequivalent drug product thereof with strong CYP3A inducers substantially decreases exposure of ivacaftor which may diminish effectiveness, and co-administration is not recommended; x) information regarding the potential for ivacaftor to affect other drugs including CYP3A and P-gp substrates, wherein the prescribing information advises caution when co-administering KALYDECO™ or bioequivalent drug product thereof with CYP3A and/or P-gp substrates. In embodiments of this aspect, the prescribing information describes CYP3A and/or P-gp substrates as selected from midazolam, alprazolam, diazepam, triaolam, digoxin, cyclosporine, and tacrolimus.

A further aspect provides a method of providing KALYDECO™ comprising: (a) providing KALYDECO™; and (b) providing product prescribing information for KALYDECO™. Yet another aspect provides for a method of providing KALYDECO™ for treating or lessening the severity of cystic fibrosis in a patient comprising: (a) providing KALYDECO™ to the patient; and providing product prescribing information for KALYDECO™ to the patient. In some embodiments, providing product prescribing information comprises providing the product prescribing information in written or electronic form.

In any of the foregoing aspects, the product prescribing information is provided as a package insert. For an example of package insert prescribing information for KALYDECO™ see http://www.accessdata.fda.gov/drugsatfda_docs/label/2012/203188s0011b1.pdf.

### III. Synthesis of Ivacaftor

Ivacaftor can be prepared by known methods. An exemplary synthesis of Ivacaftor is shown in the examples below and in Schemes 1-4, 1-5, 1-6, and 1-7. The synthesis of Ivacaftor is further described in U.S. patent application publication numbers US 2006/0074075, US 2011/0064811, US 2010/0267768, and US 2011/0230519, the contents of which are hereby incorporated by reference in their entirety.

The following is an exemplary synthesis for producing Ivacaftor, which includes the synthesis a coupling of an acid moiety and an amine moiety.

### Synthesis of the Acid Moiety

The synthesis of the acid moiety 4-Oxo-1,4-dihydroquinoline-3-carboxylic acid 26, is summarized in Scheme 1-4.

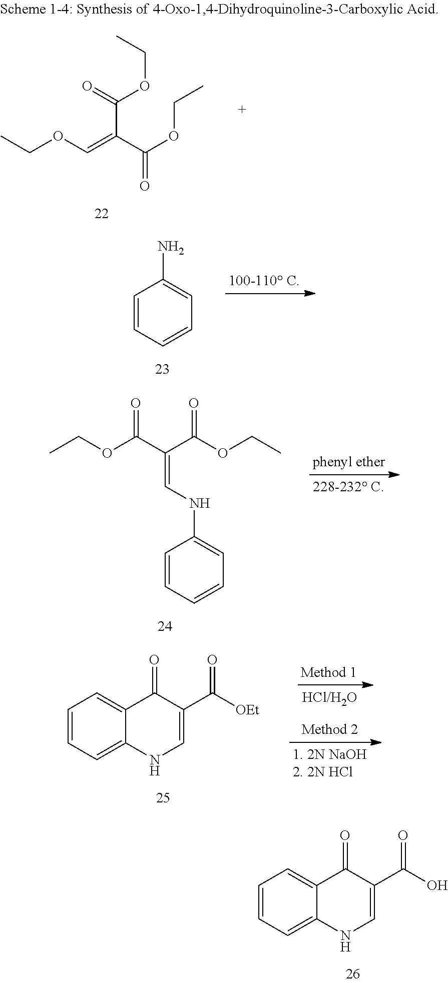 [SMILES: CCOC=C(C(=O)OCC)C(=O)OCC ; Nc1ccccc1 ; CCOC(=O)C(=CNc1ccccc1)C(=O)OCC ; CCOC(=O)c1c[nH]c2ccccc2c1=O ; O=C(O)c1c[nH]c2ccccc2c1=O]

### Example 1a

### Ethyl 4-oxo-1,4-dihydroquinoline-3-carboxylate (25)

Compound 23 (4.77 g, 47.7 mmol) was added dropwise to Compound 22 (10 g, 46.3 mmol) with subsurface N₂ flow to drive out ethanol below 30° C. for 0.5 hours. The solution was then heated to 100-110° C. and stirred for 2.5 hours. After cooling the mixture to below 60° C., diphenyl ether was added. The resulting solution was added dropwise to diphenyl ether that had been heated to 228-232° C. for 1.5 hours with subsurface N₂ flow to drive out ethanol. The mixture was stirred at 228-232° C. for another 2 hours, cooled to below 100° C. and then heptane was added to precipitate the product. The resulting slurry was stirred at 30° C. for 0.5 hours. The solids were then filtered, and the cake was washed with heptane and dried in vacuo to give Compound 25 as a brown solid. ¹H NMR (DMSO-d₆; 400 MHz) δ 12.25 (s), δ 8.49 (d), δ 8.10 (m), δ 7.64 (m), δ 7.55 (m), δ 7.34 (m), δ 4.16 (q), δ 1.23 (t).

### Example 1b

### 4-Oxo-1,4-dihydroquinoline-3-carboxylic acid (26)

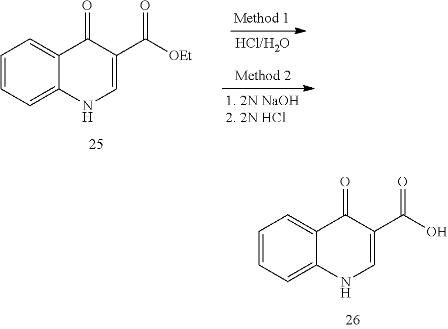 [SMILES: CCOC(=O)c1c[nH]c2ccccc2c1=O ; O=C(O)c1c[nH]c2ccccc2c1=O]

### Method 1

Compound 25 (1.0 eq) was suspended in a solution of HCl (10.0 eq) and H₂O (11.6 vol). The slurry was heated to 85-90° C., although alternative temperatures are also suitable for this hydrolysis step. For example, the hydrolysis can alternatively be performed at a temperature of from about 75 to about 100° C. In some instances, the hydrolysis is performed at a temperature of from about 80 to about 95° C. In others, the hydrolysis step is performed at a temperature of from about 82 to about 93° C. (e.g., from about 82.5 to about 92.5° C. or from about 86 to about 89° C.). After stirring at 85-90° C. for approximately 6.5 hours, the reaction was sampled for reaction completion. Stirring may be performed under any of the temperatures suited for the hydrolysis. The solution was then cooled to 20-25° C. and filtered. The reactor/cake was rinsed with H₂O (2 vol×2). The cake was then washed with 2 vol H₂O until the pH≧3.0. The cake was then dried under vacuum at 60° C. to give Compound 26.

### Method 2

Compound 25 (11.3 g, 52 mmol) was added to a mixture of 10% NaOH (aq) (10 mL) and ethanol (100 mL). The solution was heated to reflux for 16 hours, cooled to 20-25° C. and then the pH was adjusted to 2-3 with 8% HCl. The mixture was then stirred for 0.5 hours and filtered. The cake was washed with water (50 mL) and then dried in vacuo to give Compound 26 as a brown solid. ¹H NMR (DMSO-d₆; 400 MHz) δ 15.33 (s), δ 13.39 (s), δ 8.87 (s), δ 8.26 (m), δ 7.87 (m), δ 7.80 (m), δ 7.56 (m).

### Synthesis of the Amine Moiety

The synthesis of the amine moiety 32, is summarized in Scheme 1-5.

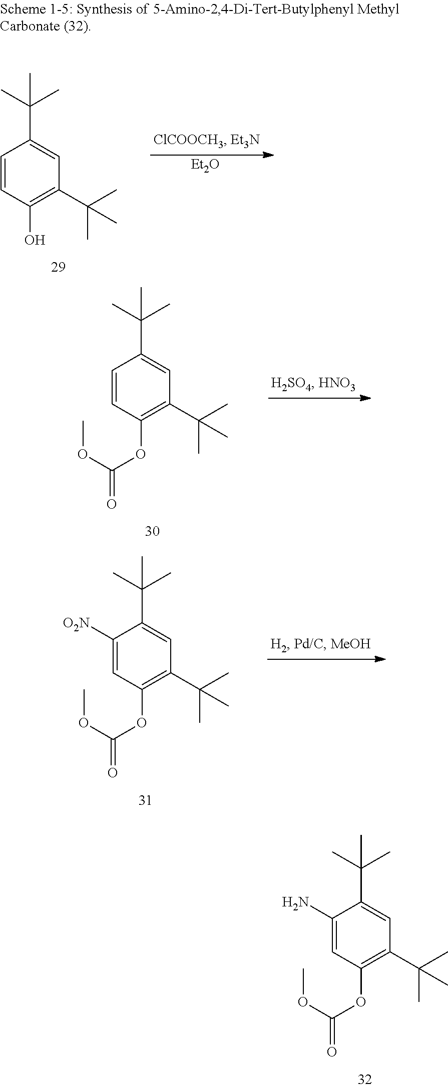 [SMILES: "Et2O" ; CC(C)(C)c1ccc(O)c(C(C)(C)C)c1 ; COC(=O)Oc1ccc(C(C)(C)C)cc1C(C)(C)C ; COC(=O)Oc1cc([N+](=O)[O-])c(C(C)(C)C)cc1C(C)(C)C ; COC(=O)Oc1cc(N)c(C(C)(C)C)cc1C(C)(C)C]

### Example 1c

### 2,4-Di-tert-butylphenyl methyl carbonate (30)

### Method 1

To a solution of 2,4-di-tert-butyl phenol (29) (10 g, 48.5 mmol) in diethyl ether (100 mL) and triethylamine (10.1 mL, 72.8 mmol), was added methyl chloroformate (7.46 mL, 97 mmol) dropwise at 0° C. The mixture was then allowed to warm to room temperature and stir for an additional 2 hours. An additional 5 mL triethylamine and 3.7 mL methyl chloroformate was then added and the reaction stirred overnight. The reaction was then filtered, the filtrate was cooled to 0° C., and an additional 5 mL triethylamine and 3.7 mL methyl chloroformate was then added and the reaction was allowed to warm to room temperature and then stir for an additional 1 hour. At this stage, the reaction was almost complete and was worked up by filtering, then washing with water (2×), followed by brine. The solution was then concentrated to produce a yellow oil and purified using column chromatography to give Compound 30. ¹H NMR (400 MHz, DMSO-d₆) δ 7.35 (d, J=2.4 Hz, 1H), 7.29 (dd, J=8.4, 2.4 Hz, 1H), 7.06 (d, J=8.4 Hz, 1H), 3.85 (s, 3H), 1.30 (s, 9H), 1.29 (s, 9H).

### Method 2

To a reactor vessel charged with 4-dimethylaminopyridine (DMAP, 3.16 g, 25.7 mmol) and 2,4-ditert-butyl phenol (Compound 29, 103.5 g, 501.6 mmol) was added methylene chloride (415 g, 313 mL) and the solution was agitated until all solids dissolved. Triethylamine (76 g, 751 mmol) was then added and the solution was cooled to 0-5° C. Methyl chloroformate (52 g, 550.3 mmol) was then added dropwise over 2.5-4 hours, while keeping the solution temperature between 0-5° C. The reaction mixture was then slowly heated to 23-28° C. and stirred for 20 hours. The reaction was then cooled to 10-15° C. and charged with 150 mL water. The mixture was stirred at 15-20° C. for 35-45 minutes and the aqueous layer was then separated and extracted with 150 mL methylene chloride. The organic layers were combined and neutralized with 2.5% HCl (aq) at a temperature of 5-20° C. to give a final pH of 5-6. The organic layer was then washed with water and concentrated in vacuo at a temperature below 20° C. to 150 mL to give Compound 30.

### Example 1d

### 5-Nitro-2,4-di-tert-butylphenyl methyl carbonate (31)

### Method 1

To a stirred solution of Compound 30 (6.77 g, 25.6 mmol) was added 6 mL of a 1:1 mixture of sulfuric acid and nitric acid at 0° C. dropwise. The mixture was allowed to warm to room temperature and stirred for 1 hour. The product was purified using liquid chromatography (ISCO, 120 g, 0-7% EtOAc/Hexanes, 38 min) producing about an 8:1-10:1 mixture of regioisomers of Compound 31 as a white solid. ¹H NMR (400 MHz, DMSO-d₆) δ 7.63 (s, 1H), 7.56 (s, 1H), 3.87 (s, 3H), 1.36 (s, 9H), 1.32 (s, 9H). HPLC ret. time 3.92 min 10-99% CH₃CN, 5 min run; ESI-MS 310 m/z (MH)⁺.

### Method 2

To Compound 30 (100 g, 378 mmol) was added DCM (540 g, 408 mL). The mixture was stirred until all solids dissolved, and then cooled to −5-0° C. Concentrated sulfuric acid (163 g) was then added dropwise, while maintaining the initial temperature of the reaction, and the mixture was stirred for 4.5 hours. Nitric acid (62 g) was then added dropwise over 2-4 hours while maintaining the initial temperature of the reaction, and was then stirred at this temperature for an additional 4.5 hours. The reaction mixture was then slowly added to cold water, maintaining a temperature below 5° C. The quenched reaction was then heated to 25° C. and the aqueous layer was removed and extracted with methylene chloride. The combined organic layers were washed with water, dried using Na₂SO₄, and concentrated to 124-155 mL. Hexane (48 g) was added and the resulting mixture was again concentrated to 124-155 mL. More hexane (160 g) was subsequently added to the mixture. The mixture was then stirred at 23-27° C. for 15.5 hours, and was then filtered. To the filter cake was added hexane (115 g), the resulting mixture was heated to reflux and stirred for 2-2.5 hours. The mixture was then cooled to 3-7° C., stirred for an additional 1-1.5 hours, and filtered to give Compound 31 as a pale yellow solid.

### Example 1e

### 5-Amino-2,4-di-tert-butylphenyl methyl carbonate (32)

2,4-Di-tert-butyl-5-nitrophenyl methyl carbonate (1.00 eq) was charged to a suitable hydrogenation reactor, followed by 5% Pd/C (2.50 wt % dry basis, Johnson-Matthey Type 37). MeOH (15.0 vol) was charged to the reactor, and the system was closed. The system was purged with N₂ (g), and was then pressurized to 2.0 Bar with H₂ (g). The reaction was performed at a reaction temperature of 25° C.+/−5° C. When complete, the reaction was filtered, and the reactor/cake was washed with MeOH (4.00 vol). The resulting filtrate was distilled under vacuum at no more than 50° C. to 8.00 vol. Water (2.00 vol) was added at 45° C.+/−5° C. The resultant slurry was cooled to 0° C.+/−5. The slurry was held at 0° C.+/−5° C. for no less than 1 hour, and filtered. The cake was washed once with 0° C.+/−5° C. MeOH/H₂O (8:2) (2.00 vol). The cake was dried under vacuum (−0.90 bar and −0.86 bar) at 35° C.-40° C. to give Compound 32. ¹H NMR (400 MHz, DMSO-d₆) δ 7.05 (s, 1H), 6.39 (s, 1H), 4.80 (s, 2H), 3.82 (s, 3H), 1.33 (s, 9H), 1.23 (s, 9H).

Once the reaction was complete, the resulting mixture was diluted with from about 5 to 10 volumes of MeOH (e.g., from about 6 to about 9 volumes of MeOH, from about 7 to about 8.5 volumes of MeOH, from about 7.5 to about 8 volumes of MeOH, or about 7.7 volumes of MeOH), heated to a temperature of about 35±5° C., and filtered to remove palladium. The reactor cake was washed before combining the filtrate and wash, distilling, adding water, cooling, filtering, washing and drying the product cake as described above.

### Coupling the Acid and Amine Moieties

The coupling of the acid moiety to the amine moiety is summarized in Scheme 1-6.

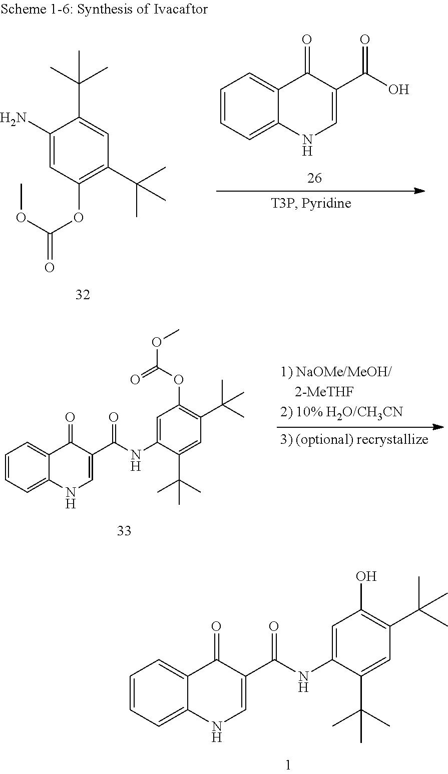 [SMILES: COC(=O)Oc1cc(N)c(C(C)(C)C)cc1C(C)(C)C ; O=C(O)c1c[nH]c2ccccc2c1=O ; COC(=O)Oc1cc(NC(=O)c2c[nH]c3ccccc3c2=O)c(C(C)(C)C)cc1C(C)(C)C ; CC(C)(C)c1cc(C(C)(C)C)c(NC(=O)c2c[nH]c3ccccc3c2=O)cc1O]

### Example 1f

### N-(2,4-di-tert-butyl-5-hydroxyphenyl)-4-oxo-1,4-dihydroquinoline-3-carboxamide (1)

4-Oxo-1,4-dihydroquinoline-3-carboxylic acid (26) (1.0 eq) and 5-amino-2,4-di-tert-butylphenyl methyl carbonate (32) (1.1 eq) were charged to a reactor. 2-MeTHF (4.0 vol, relative to the acid) was added followed by T3P® 50% solution in 2-MeTHF (1.7 eq). The T3P charged vessel was washed with 2-MeTHF (0.6 vol). Pyridine (2.0 eq) was then added, and the resulting suspension was heated to 47.5+/−5.0° C. and held at this temperature for 8 hours. A sample was taken and checked for completion by HPLC. Once complete, the resulting mixture was cooled to 25.0° C.+/−2.5° C. 2-MeTHF was added (12.5 vol) to dilute the mixture. The reaction mixture was washed with water (10.0 vol) 2 times. 2-MeTHF was added to bring the total volume of reaction to 40.0 vol (˜16.5 vol charged). To this solution was added NaOMe/MeOH (1.7 equiv) to perform the methanolysis. The reaction was stirred for no less than 1.0 hour, and checked for completion by HPLC. Once complete, the reaction was quenched with 1N HCl (10.0 vol), and washed with 0.1N HCl (10.0 vol). The organic solution was polish filtered to remove any particulates and placed in a second reactor. The filtered solution was concentrated at no more than 45° C. (jacket temperature) and no less than 8.0° C. (internal reaction temperature) under reduced pressure to 20 vol. CH₃CN was added to 40 vol and the solution concentrated at no more than 45° C. (jacket temperature) and no less than 8.0° C. (internal reaction temperature) to 20 vol. The addition of CH₃CN and concentration cycle was repeated 2 more times for a total of 3 additions of CH₃CN and 4 concentrations to 20 vol. After the final concentration to 20 vol, 16.0 vol of CH₃CN was added followed by 4.0 vol of H₂O to make a final concentration of 40 vol of 10% H₂O/CH₃CN relative to the starting acid. This slurry was heated to 78.0° C.+/−5.0° C. (reflux). The slurry was then stirred for no less than 5 hours. The slurry was cooled to 0.0° C.+/−5° C. over 5 hours, and filtered. The cake was washed with 0.0° C.+/−5.0° C. CH₃CN (5 vol) 4 times. The resulting solid (Ivacaftor) was dried in a vacuum oven at no more than 50.0° C. ¹H NMR (400 MHz, DMSO-d₆) δ 12.8 (s, 1H), 11.8 (s, 1H), 9.2 (s, 1H), 8.9 (s, 1H), 8.3 (s, 1H), 7.2 (s, 1H), 7.9 (t, 1H), 7.8 (d, 1H), 7.5 (t, 1H), 7.1 (s, 1H), 1.4 (s, 9H), 1.4 (s, 9H).

An alternative synthesis of Ivacaftor is depicted in Scheme 1-7.

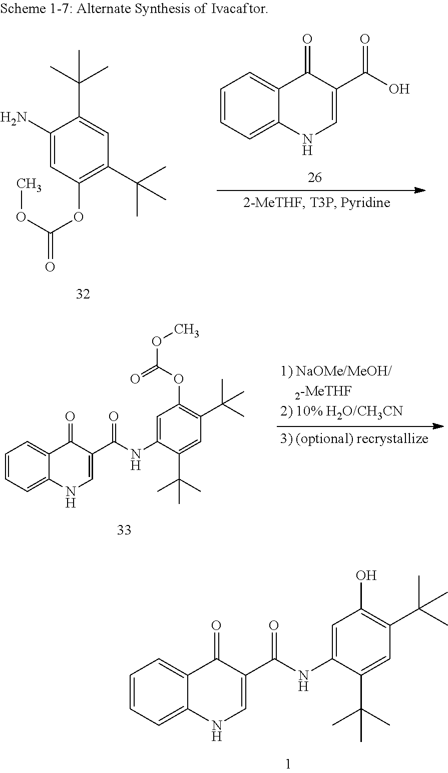 [SMILES: COC(=O)Oc1cc(N)c(C(C)(C)C)cc1C(C)(C)C ; O=C(O)c1c[nH]c2ccccc2c1=O ; COC(=O)Oc1cc(NC(=O)c2c[nH]c3ccccc3c2=O)c(C(C)(C)C)cc1C(C)(C)C ; CC(C)(C)c1cc(C(C)(C)C)c(NC(=O)c2c[nH]c3ccccc3c2=O)cc1O]

### Example 1g

### N-(2,4-di-tert-butyl-5-hydroxyphenyl)-4-oxo-1,4-dihydroquinoline-3-carboxamide (1)

4-Oxo-1,4-dihydroquinoline-3-carboxylic acid 26 (1.0 eq) and 5-amino-2,4-di-tert-butylphenyl methyl carbonate 32 (1.1 eq) were charged to a reactor. 2-MeTHF (4.0 vol, relative to the acid) was added followed by T3P® 50% solution in 2-MeTHF (1.7 eq). The T3P charged vessel was washed with 2-MeTHF (0.6 vol). Pyridine (2.0 eq) was then added, and the resulting suspension was heated to 47.5+/−5.0° C. and held at this temperature for 8 hours. A sample was taken and checked for completion by HPLC. Once complete, the resulting mixture was cooled to 20° C.+/−5° C. 2-MeTHF was added (12.5 vol) to dilute the mixture. The reaction mixture was washed with water (10.0 vol) 2 times and 2-MeTHF (16.5 vol) was charged to the reactor. This solution was charged with 30% w/w NaOMe/MeOH (1.7 equiv) to perform the methanolysis. The reaction was stirred at 25.0° C.+/−5.0° C. for no less than 1.0 hour, and checked for completion by HPLC. Once complete, the reaction was quenched with 1.2 N HCl/H₂O (10.0 vol), and washed with 0.1N HCl/H₂O (10.0 vol). The organic solution was polish filtered to remove any particulates and placed in a second reactor.

The filtered solution was concentrated at no more than 45° C. (jacket temperature) and no less than 8.0° C. (internal reaction temperature) under reduced pressure to 20 vol. CH₃CN was added to 40 vol and the solution concentrated at no more than 45° C. (jacket temperature) and no less than 8.0° C. (internal reaction temperature) to 20 vol. The addition of CH₃CN and concentration cycle was repeated 2 more times for a total of 3 additions of CH₃CN and 4 concentrations to 20 vol. After the final concentration to 20 vol, 16.0 vol of CH₃CN was charged followed by 4.0 vol of H₂O to make a final concentration of 40 vol of 10% H₂O/CH₃CN relative to the starting acid. This slurry was heated to 78.0° C.+/−5.0° C. (reflux). The slurry was then stirred for no less than 5 hours. The slurry was cooled to 20 to 25° C. over 5 hours, and filtered. The cake was washed with CH₃CN (5 vol) heated to 20 to 25° C. 4 times. The resulting solid (Ivacaftor) was dried in a vacuum oven at no more than 50.0° C. ¹H NMR (400 MHz, DMSO-d₆) δ 12.8 (s, 1H), 11.8 (s, 1H), 9.2 (s, 1H), 8.9 (s, 1H), 8.3 (s, 1H), 7.2 (s, 1H), 7.9 (t, 1H), 7.8 (d, 1H), 7.5 (t, 1H), 7.1 (s, 1H), 1.4 (s, 9H), 1.4 (s, 9H).

### IV. Formulations of Ivacaftor

Ivacaftor can be formulated as the commercially approved drug product KALYDECO™. Solid dispersions and pharmaceutical compositions of Ivacaftor that are useful in producing KALYDECO™ are further described in the U.S. patent application publications US 2011/0064811, US 2010/0074949, and US 2010/0256184, the contents of which are hereby incorporated by reference in their entirety.

KALYDECO™ is a caplet shaped pharmaceutical tablet composition comprising about 34.1 wt % of a solid dispersion by weight of the composition, wherein the dispersion comprises about 80 wt % of substantially amorphous Ivacaftor by weight of the dispersion, about 19.5 wt % of HPMCAS by weight of the dispersion, and about 0.5 wt % SLS by weight of the dispersion; about 30.5 wt % of microcrystalline cellulose by weight of the composition; about 30.4 wt % of lactose by weight of the composition; about 3 wt % of sodium croscarmellose by weight of the composition; about 0.5 wt % of SLS by weight of the composition; about 0.5 wt % of colloidal silicon dioxide by weight of the composition; and about 1 wt % of magnesium stearate by weight of the composition. The caplet shaped pharmaceutical tablet composition comprises a colorant coated, a wax coating, and a printed logo or text. Although caplet shaped pharmaceutical tablets can be produced with different amounts of Ivacaftor (e.g., 75 mg, 100 mg, 150 mg, etc.), the caplet shaped pharmaceutical tablet composition for KALYDECO™ contains 150 mg of Ivacaftor per tablet.

### Exemplary Preparations of KALYDECO™

The following provides an exemplary method for producing KALYDECO™

A solvent system of MEK and DI water, formulated according to the ratio 90 wt % MEK/10 wt % DI water, was heated to a temperature of 20-30° C. in a reactor, equipped with a magnetic stirrer and thermal circuit. Into this solvent system, hypromellose acetate succinate polymer (HPMCAS)(HG grade), SLS, and Ivacaftor were added according to the ratio 19.5 wt % hypromellose acetate succinate/0.5 wt % SLS/80 wt % Ivacaftor. The resulting mixture contained 10.5 wt % solids. The actual amounts of ingredients and solvents used to generate this mixture are recited in Table 2-F1.

TABLE 2-F1
Solid Spray Dispersion Ingredients for Intermediate F.

| | Units | Batch |
|---|---|---|
| Ivacaftor | Kg | 70.0 |
| HPMCAS | Kg | 17.1 |
| SLS | Kg | 0.438 |
| Total Solids | Kg | 87.5 |
| MEK | Kg | 671 |
| Water | Kg | 74.6 |
| Total Solvents | Kg | 746 |
| Total Spray Solution Weight | Kg | 833 |

The mixture temperature was adjusted to a range of 20-45° C. and mixed until it was substantially homogenous and all components were substantially dissolved.

A spray drier, Niro PSD4 Commercial Spray Dryer, fitted with pressure nozzle (Spray Systems Maximum Passage series SK-MFP having orifice/core size 54/21) equipped with anti-bearding cap, was used under normal spray drying mode, following the dry spray process parameters recited in Table 2-F2.

TABLE 2-F2
Dry Spray Process Parameters Used to Generate Intermediate F.

| Parameter | Value |
|---|---|
| Feed Pressure | 20 bar |
| Feed Flow Rate | 92-100 Kg/hr |
| Inlet Temperature | 93-99° C. |
| Outlet Temperature | 53-57° C. |
| Vacuum Dryer Temperature | 80° C. for 2 hours then |
| | 110° C. (+/−5° C.) |
| Vacuum Drying Time | 20-24 hours |

A high efficiency cyclone separated the wet product from the spray gas and solvent vapors. The wet product contained 8.5-9.7% MEK and 0.56-0.83% Water and had a mean particle size of 17-19 um and a bulk density of 0.27-0.33 g/cc. The wet product was transferred to a 4000L stainless steel double cone vacuum dryer for drying to reduce residual solvents to a level of less than about 5000 ppm and to generate dry Intermediate F. The dry Intermediate F contained <0.03% MEK and 0.3% Water.

Intermediate G

A solvent system of MEK and DI water, formulated according to the ratio 90 wt % MEK/10 wt % DI water, was heated to a temperature of 20-30° C. in a reactor, equipped with a magnetic stirrer and thermal circuit. Into this solvent system, hypromellose acetate succinate polymer (HPMCAS)(HG grade), SLS, and Ivacaftor were added according to the ratio 19.5 wt % hypromellose acetate succinate/0.5 wt % SLS/80 wt % Ivacaftor. The resulting mixture contained 10.5 wt % solids. The actual amounts of ingredients and solvents used to generate this mixture are recited in Table 2-G1.

TABLE 2-G1
Solid Spray Dispersion Ingredients for Intermediate G.

| | Units | Batch |
|---|---|---|
| Ivacaftor | Kg | 24.0 |
| HPMCAS | Kg | 5.85 |
| SLS | Kg | 0.15 |
| Total Solids | Kg | 30.0 |
| MEK | Kg | 230.1 |
| Water | Kg | 25.6 |
| Total Solvents | Kg | 255.7 |
| Total Spray Solution Weight | Kg | 285.7 |

The mixture temperature was adjusted to a range of 20-45° C. and mixed until it was substantially homogenous and all components were substantially dissolved.

A spray drier, Niro Production Minor Spray Dryer, fitted with pressure nozzle (Spray Systems Maximum Passage series SK-MFP having orifice size 72) was used under normal spray drying mode, following the dry spray process parameters recited in Table 2-G2.

TABLE 2-G2
Dry Spray Process Parameters Used to Generate Intermediate G.

| Parameter | Value |
|---|---|
| Feed Pressure | 33 bar |
| Feed Flow Rate | 18-24 Kg/hr |
| Inlet Temperature | 82-84° C. |
| Outlet Temperature | 44-46° C. |
| Vacuum Dryer Temperature | 80° C. for 2 hours then |
| | 110° C. (+/−5° C.) |
| Vacuum Drying Time | 48 hours |

A high efficiency cyclone separated the wet product from the spray gas and solvent vapors. The wet product contained 10.8% MEK and 0.7% Water and had a mean particle size of 19 um and a bulk density of 0.32 g/cc. The wet product was transferred to a 4000L stainless steel double cone vacuum dryer for drying to reduce residual solvents to a level of less than about 5000 ppm and to generate dry Intermediate. The dry Intermediate G contained <0.05% MEK and 0.7% Water.

Intermediate H

A solvent system of MEK and DI water, formulated according to the ratio 90 wt % MEK/10 wt % DI water, was heated to a temperature of 20-30° C. in a reactor, equipped with a magnetic stirrer and thermal circuit. Into this solvent system, hypromellose acetate succinate polymer (HPMCAS)(HG grade), SLS, and Ivacaftor were added according to the ratio 19.5 wt % hypromellose acetate succinate/0.5 wt % SLS/80 wt % Ivacaftor. The actual amounts of ingredients and solvents used to generate this mixture are recited in Table 2-H1:

TABLE 2-H1
Solid Spray Dispersion Ingredients for Intermediate H.

| | Units | Batch |
|---|---|---|
| Ivacaftor | Kg | 56.0 |
| HPMCAS | Kg | 13.65 |
| SLS | Kg | 0.35 |
| Total Solids | Kg | 70.0 |
| MEK | Kg | 509.73 |
| Water | Kg | 56.64 |
| Total Solvents | Kg | 566.40 |
| Total Spray Solution Weight | Kg | 636.40 |

The mixture temperature was adjusted to a range of 20-30° C. and mixed until it was substantially homogenous and all components were substantially dissolved.

A spray drier, Niro Production Minor Spray Dryer, fitted with pressure nozzle (Spray Systems Maximum Passage series SK-MFP having orifice size #52 or #54, e.g., about 1.39-1.62 mm) was used under normal spray drying mode, following the dry spray process parameters recited in Table 2-H2.

TABLE 2-H2
Dry Spray Process Parameters Used to Generate Intermediate H.

| Parameter | Value |
|---|---|
| Feed Pressure | 20-50 bar |
| Feed Flow Rate | 18-24 Kg/hr |
| Inlet Temperature | −7 to 7° C. |
| Outlet Temperature | 30-70° C. |

A high efficiency cyclone separated the wet product from the spray gas and solvent vapors. The wet product contained approximately 10.8% MEK and 0.7% Water and had a mean particle size of about 19 μm and a bulk density of about 0.33 g/cc.

An inertial cyclone is used to separate the spray dried intermediate from the process gas and solvent vapors. Particle size is monitored on-line. The spray dried intermediate is collected in an intermediate bulk container. The process gas and solvent vapors are passed through a filter bag to collect the fine particles not separated by the cyclone. The resultant gas is condensed to remove process vapors and recycled back to the heater and spray dryer. The spray dried intermediate will be stored at less than 30° C., if secondary drying will occur in less than 24 hours or between 2-8° C., if secondary drying will occur in more than 24 hours.

Secondary drying occurs by charging a 4000-L biconical dryer having a jacket temperature between about 20-30° C. with the spray dried intermediate. The vacuum pressure, jacket temperature, and nitrogen bleed are set at between about −0.8 psig and about −1.0 psig, between about 80-120° C., and between about 0.5-8.0 m³/h, respectively. Agitation is set at 1 rpm. Bulk samples of the spray dried intermediate are tested for MEK (GC), every 4 hours until dry. The MEK drying rate is monitored on-line by GC-MS, calibrated for MEK concentration. Upon reaching a plateau in the drying of the residual MEK, heating in the biconical dryer is discontinued while continuing rotation until the spray dried intermediate reaches a temperature less than or equal to 50° C.

Although Intermediates F through H are described above as being formed, in part, by admixing the solid spray dispersion ingredients with application of heat to form a homogeneous mixture, the solid spray dispersion ingredients can also be mixed without application of heat to form a mixture of the solid spray dispersion ingredients.

### Exemplary KALYDECO™ Tablet Containing 150 Mg of Ivacaftor

A batch of caplet-shaped tablets was formulated to have about 150 mg of Ivacaftor per tablet using the amounts of ingredients recited in Table 3-10.

TABLE 3-10
Ingredients for Exemplary Tablet 11.

| | Percent Dose | Dose | Batch |
|---|---|---|---|
| Tablet Formulation | % Wt./Wt. | (mg) | (g) |
| Intermediate F | 34.09% | 187.5 | 23.86 |
| Microcrystalline cellulose | 30.51% | 167.8 | 21.36 |
| Lactose | 30.40% | 167.2 | 21.28 |
| Sodium croscarmellose | 3.000% | 16.50 | 2.100 |
| SLS | 0.500% | 2.750 | 0.3500 |
| Colloidal silicon dioxide | 0.500% | 2.750 | 0.3500 |
| Magnesium stearate | 1.000% | 5.500 | 0.7000 |
| Total | 100% | 550 | 70 |

The colloidal silicon dioxide (Cabot Cab-O—Sil® M-5P Fumed Silicon Dioxide) and the microcrystalline cellulose (FMC MCC Avicel® PH102) were passed through a 30 mesh screen.

The sodium croscarmellose (FMC Ac-Di-Sol®), SLS, Intermediate F, and lactose (Foremost FastFlo® Lactose #316) were also passed, individually in the preceding order, through the same 30 mesh screen. A nitrogen purge was used when screening Intermediate F. The screened components were loaded into a 10 cubic feet V-blender, which was purged with nitrogen, and blended for about 180 (+/−10) inversions.

The Magnesium Stearate was filtered through a 40 mesh screen sieve into the blending container and mixed to provide about 54 inversions.

The resulting mixture was compressed into tablets using a fully tooled 36 Fette 2090 press with 0.568″×0.2885″ caplet type B tooling set to produce a tablet having an initial target hardness of about 10 Kp±20%.

A batch of caplet-shaped tablets from above was spray-coated with OPADRY® II (Blue, Colorcon) to a weight gain of about 3.0% using a 24″ coating pan configured with the parameters in Table 3-11 followed by wax coating and then printing using Opacode® S-1-17823 (Solvent based Black, Colorcon).

TABLE 3-11
Spray-Coating Process Parameters

| Coating Parameters 24″ Pan | Target |
|---|---|
| Pan Load (kg) | 14 |
| Inlet Temperature (° C.)* | * |
| Pan Speed (rpm) | 10 |
| Jog Time (sec) | 2-5 sec every |
| | 60 sec |
| # of Spray Guns | 2 |
| Solids Content (% w/w) | 20 |
| Gun to Bed Distance (inches) | 6 |
| Inlet Air Flow (cfm) | 300 |
| Spray Rate (g/min) | 35 |
| Exhaust Temperature (° C.) | 50 |
| Atomization Pressure (psi) | 42 |

*Inlet temperature is monitored to achieve target exhaust temperature. Initial inlet temperature should be set at about 75° C. to achieve target exhaust temp.

The OPADRY® II suspension was prepared by measuring an amount of de-ionized water which when combined with OPADRY® II would produce a total solids content of 20% w/w. The water is mixed to a vortex followed by addition of OPADRY® II over a period of approximately 5 minutes. Once the OPADRY® II powder was wetted, mixing was continued to ensure that all solid material is well-dispersed. The suspension is then charged into a Thomas 24″ pan coating instrument using coating conditions outlined in Table 3-11.

Uncoated tablets are placed into the coating pan and pre-warmed. The inlet was increased from room temperature to about 55° C. and then increased as necessary to provide the exhaust temperature in Table 3-11. The coating process was performed with 20% w/w OPADRY® II (85 Series Blue) coating dispersion to obtain a target weight gain of about 3%. The coated tablets were then allowed to tumble for about 2 minutes without spraying. The bed temperature was then allowed to cool to about 35° C.

Upon cooling, the Carnauba wax powder was weighed out in the amount of about 0.01% w/w of the starting tablet core weight. With the air flow off, the carnauba wax powder was sprinkled evenly on the tablet bed. The pan bed was turned on to the speed indicated in Table 3-11. After 5 minutes, the air flow was turned on (without heating) to the setting indicated in Table 3-11. After about one minute, the air flow and pan were turned off.

Once coated with OPADRY® II, the tablets are then labeled using a Hartnett Delta tablet printer charged with Opacode® S-1-17823.

### Another Exemplary KALYDECO™ Tablet Containing 150 Mg of Ivacaftor

A batch of caplet-shaped tablets is formulated to have about 150 mg of Ivacaftor per tablet using the amounts of ingredients recited in Table 3-12.

TABLE 3-12
Ingredients for Exemplary Tablet 13.

| | Percent Dose |
|---|---|
| Tablet Formulation | % Wt./Wt. |
| Intermediate H | 34.1% |
| Microcrystalline cellulose | 30.5% |
| Lactose | 30.4% |
| Sodium croscarmellose | 3.000% |
| SLS | 0.500% |
| Colloidal silicon dioxide | 0.500% |
| Magnesium stearate | 1.000% |
| Total | 100% |

The colloidal silicon dioxide (Cabot Cab-O—Sil® M-5P Fumed Silicon Dioxide) and the microcrystalline cellulose (FMC MCC Avicel® PH102) are passed through a 30 mesh screen.

The sodium croscarmellose (FMC Ac-Di-Sol®), SLS, Intermediate H, and lactose (Foremost FastFlo® Lactose #316) are also passed, individually in the preceding order, through the same 30 mesh screen. A nitrogen purge is used when screening Intermediate H. The screened components are loaded into a 10 cubic feet V-blender, which is purged with nitrogen, and blended for about 180 (+/−10) inversions.

The Magnesium Stearate is filtered through a 40 mesh screen sieve into the blending container and mixed to provide about 54 inversions.

The resulting mixture is compressed into tablets using a fully tooled 36 Fette 2090 press with 0.568″×0.2885″ caplet type B tooling set to produce a tablet having an initial target hardness of about 10 Kp±20%.

A batch of caplet-shaped tablets from above is spray-coated with OPADRY® II (Blue, Colorcon) to a weight gain of about 3.0% using a Thomas 48″ coating pan configured with the parameters in Table 3-13 followed by wax coating and then printing using Opacode® S-1-17823 (Solvent based Black, Colorcon).

TABLE 3-13
Spray-Coating Process Parameters

| Coating Parameters 48″ Pan | Target |
|---|---|
| Pan Load (kg) | up to 120 |
| Inlet Temperature (° C.)* | * |
| # of Spray Guns | 4 |
| Solids Content (% w/w) | 20 |
| Gun to Bed Distance (inches) | 7-7.5 |
| Inlet Air Flow (cfm) | 1050-2400 |
| Spray Rate (ml/min) | 203-290 |
| Exhaust Temperature (° C.) | 40-65 |
| Atomization Pressure (slpm) | 145 |

*Inlet temperature is monitored to achieve target exhaust temperature. Initial inlet temperature should be set at about 50-75° C. to achieve target exhaust temp.

The OPADRY® II suspension is prepared by measuring an amount of de-ionized water which when combined with OPADRY® II would produce a total solids content of 20% w/w. The water is mixed to a vortex followed by addition of OPADRY® II over a period of approximately 5 minutes. Once the OPADRY® II powder is wetted, mixing is continued to ensure that all solid material is well-dispersed. The suspension is then charged into a Thomas 48″ pan coating instrument using coating conditions outlined in Table 3-13. In other examples, the suspension can be coated with a Thomas 24″ pan coating instrument.

Uncoated tablets are placed into the coating pan and pre-warmed. The inlet is increased from room temperature to about 55° C. and then increased as necessary to provide the exhaust temperature in Table 3-13. The coating process is performed with 20% w/w OPADRY® II (85 Series Blue) coating dispersion to obtain a target weight gain of about 3%. The coated tablets are then allowed to tumble for about 2 minutes without spraying. The bed temperature is then allowed to cool to about 35° C.

Upon cooling, the Carnauba wax powder is weighed out in the amount of about 0.01% w/w of the starting tablet core weight. With the air flow off, the carnauba wax powder is sprinkled evenly on the tablet bed. The pan bed is turned on to the speed indicated in Table 3-13. After 5 minutes, the air flow is turned on (without heating) to the setting indicated in Table 3-13. After about one minute the air flow and pan is turned off.

Once coated with OPADRY® II, the tablets are then labeled using a Hartnett Delta tablet printer charged with Opacode® S-1-17823.

### V. Prescribing Information for KALYDECO™

1. Indications and Usage

KALYDECO™ is classified as a cystic fibrosis transmembrane conductance regulator (CFTR) potentiator. KALYDECO™ is indicated for the treatment of cystic fibrosis (CF) in patients age 6 years and older who have a G551D mutation in the CFTR gene. If the patient's genotype is unknown, an FDA-cleared CF mutation test should be used to detect the presence of the G551D mutation.

Limitations of Use

KALYDECO™ is not effective in patients with CF who are homozygous for the F508del mutation in the CFTR gene and has not been studied in other populations of patients with CF.

2. Dosage and Administration

2.1 Dosing Information in Adults and Children Ages 6 Years and Older

The recommended dose of KALYDECO™ for both adults and pediatric patients age 6 years and older is one 150 mg tablet taken orally every 12 hours (300 mg total daily dose) with fat-containing food. Examples of appropriate fat-containing food include eggs, butter, peanut butter, cheese pizza, etc. [see Clinical Pharmacology (subsection 12.3 of section 9 and Patient Counseling Information (subsection 17.4 of section V)].

2.2 Dosage Adjustment for Patients with Hepatic Impairment

In patients with moderate or severe hepatic impairment, the dose should be reduced. The dose of KALYDECO™ should be reduced to 150 mg once daily for patients with moderate hepatic impairment (Child-Pugh Class B). KALYDECO™ should be used with caution in patients with severe hepatic impairment (Child-Pugh Class C) at a dose of 150 mg once daily or less frequently [see Use in Specific Populations (subsection 8.6 of section V), Clinical Pharmacology (subsection 12.3 of section V), and Patient Counseling Information (subsection 17.3 of section V)].

2.3 Dosage Adjustment for Patients Taking Drugs that are CYP3A Inhibitors

When co-administered with drugs that are moderate or strong CYP3A inhibitors, the dose should be reduced. When KALYDECO™ is being co-administered with strong CYP3A inhibitors (e.g., ketoconazole), the dose should be reduced to 150 mg twice-a-week. The dose of KALYDECO™ should be reduced to 150 mg once daily when co-administered with moderate CYP3A inhibitors (e.g., fluconazole). Food containing grapefruit or Seville oranges should be avoided [see Drug Interactions (subsection 7.1 of section V), Clinical Pharmacology (subsection 12.3 of section V), and Patient Counseling Information (subsection 17.2 of section V)].

3. Dosage Forms and Strengths

150 mg tablets.

4. Contraindications

None known.

5. Warnings and Precautions

5.1 Transaminase (ALT or AST) Elevations

Elevated transaminases have been reported in patients with CF receiving KALYDECO™. It is recommended that transamimases (ALT and AST) be assessed prior to initiating KALYDECO™, every 3 months during the first year of treatment, and annually thereafter. Patients who develop increased transaminase levels should be closely monitored until the abnormalities resolve. Dosing should be interrupted in patients with ALT or AST of greater than 5 times the upper limit of normal (ULN). Following resolution of transaminase elevations, consider the benefits and risks of resuming KALYDECO™ dosing [see Adverse Reactions (subsection 6 of section V)].

5.2 Concomitant Use with CYP3A Inducers

Use of KALYDECO™ with strong CYP3A inducers, such as rifampin, substantially decreases the exposure of ivacaftor, which may reduce the therapeutic effectiveness of KALYDECO™. Therefore, co-administration of KALYDECO™ with strong CYP3A inducers (e.g., rifampin, St. John's Wort) is not recommended [see Drug Interactions (subsection 7.2 of section V) and Clinical Pharmacology (subsection 12.3 of section V)].

6. Adverse Reactions

The following adverse reaction is discussed in greater detail in other sections of the label: Transaminase Elevations [see Warnings and Precautions (subsection 5.1 of section V)]

6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in clinical practice.

The overall safety profile of KALYDECO™ is based on pooled data from placebo-controlled clinical trials conducted in 353 patients with CF who had a G551D mutation in the CFTR gene or were homozygous for the F508del mutation. Of the 353 patients, 50% of patients were female and 97% were Caucasian; 221 received KALYDECO™ and 132 received placebo from 16 to 48 weeks. Patients treated with KALYDECO™ were between the ages of 6 and 53 years.

In these trials, the proportion of patients who prematurely discontinued study drug due to adverse reactions was 2% for KALYDECO™-treated patients and 5% for placebo-treated patients. Serious adverse reactions, whether considered drug-related or not by the investigators, which occurred more frequently in KALYDECO™-treated patients included abdominal pain, increased hepatic enzymes, and hypoglycemia.

Overall, the most common adverse reactions in 221 patients with CF who had either a G551D mutation or were homozygous for the F508del mutation in the CFTR gene and treated with KALYDECO™ were headache (17%), upper respiratory tract infection (16%), nasal congestion (16%), nausea (10%), rash (10%), rhinitis (6%), dizziness (5%), arthralgia (5%), and bacteria in sputum (5%).

The incidence of adverse reactions below is based upon two double-blind, placebo-controlled 48-week clinical trials in a total of 213 patients with CF ages 6 to 53 who have a G551D mutation in the CFTR gene and who were treated with KALYDECO™ 150 mg orally or placebo twice daily. Table 4 shows adverse reactions occurring in >8% of KALYDECO™-treated patients with CF who have a G551D mutation in the CFTR gene that also occurred at a higher rate than in the placebo-treated patients in the two double-blind, placebo-controlled trials.

TABLE 4
Incidence of Adverse Drug Reactions in ≧8% of
KALYDECO™-Treated Patients with a G551D Mutation in the
CFTR Gene and Greater than Placebo in 2 Placebo-Controlled
Phase 3 Clinical Trials of 48 Weeks Duration

Incidence: Pooled
48-week Trials

| | KALYDECO | Placebo |
|---|---|---|
| Adverse Reaction | N = 109 | N = 104 |
| (Preferred Term) | n (%) | n (%) |
| Headache | 26 (24) | 17 (16) |
| Oropharyngeal pain | 24 (22) | 19 (18) |
| Upper respiratory tract | 24 (22) | 14 (14) |
| infection | | |
| Nasal congestion | 22 (20) | 16 (15) |
| Abdominal pain | 17 (16) | 13 (13) |
| Nasopharyngitis | 16 (15) | 12 (12) |
| Diarrhea | 14 (13) | 10 (10) |
| Rash | 14 (13) | 7 (7) |
| Nausea | 13 (12) | 11 (11) |
| Dizziness | 10 (9) | 1 (1) |

Adverse reactions that occurred in the KALYDECO™ group at a frequency of 4 to 7% where rates exceeded that in the placebo group include:
Infections and infestations: rhinitis
Investigations: aspartate aminotransferase increased, bacteria in sputum, blood glucose increased, hepatic enzyme increased
Musculoskeletal and connective tissue disorders: arthralgia, musculoskeletal chest pain, myalgia
Nervous system disorders: sinus headache
Respiratory, thoracic and mediastinal disorders: pharyngeal erythema, pleuritic pain, sinus congestion, wheezing
Skin and subcutaneous tissue disorders: acne
Upper respiratory tract infection may include sore throat, nasal or sinus infection and/or runny nose.

Laboratory Abnormalities

Transaminase Elevations:

During 48-week, placebo-controlled clinical studies, the incidence of maximum transaminase (ALT or AST)>8, >5 or >3×ULN was 2%, 3% and 6% in KALYDECO™-treated patients and 2%, 2% and 8% in placebo-treated patients, respectively. Two patients (2%) on placebo and 1 patient (0.5%) on KALYDECO™ permanently discontinued treatment for elevated transaminases, all >8×ULN. Two patients treated with KALYDECO™ were reported to have serious adverse reactions of elevated liver transaminases compared to none on placebo [see Warnings and Precautions (subsection 5.1 of section V)].

Upper respiratory infection or common cold includes but is not limited to, sore throat, nasal or sinus congestion or runny nose.

7. Drug Interactions

Potential for Other Drugs to Affect Ivacaftor

7.1 Inhibitors of CYP3A

Ivacaftor is a sensitive CYP3A substrate. Co-administration with ketoconazole, a strong CYP3A inhibitor, significantly increased ivacaftor exposure [measured as area under the curve (AUC)] by 8.5-fold. Therefore, a reduction of the KALYDECO™ dose to 150 mg twice-a-week is recommended for co-administration with strong CYP3A inhibitors, such as ketoconazole, itraconazole, posaconazole, voriconazole, telithromycin, and clarithromycin.

Strong CYP3A inhibitors include, but are not limited to: (1) antifungal medications such as ketoconazole (e.g., Nizoral®), itraconazole (e.g., Sporanox®), posaconazole (e.g., Noxafil®), or voriconazole (e.g., Vfend®); or (2) antibiotics such as telithromycin (e.g., Ketek®), or clarithromycin (e.g., Biaxin®).

Co-administration with fluconazole, a moderate inhibitor of CYP3A, increased ivacaftor exposure by 3-fold. Therefore, a reduction of the KALYDECO™ dose to 150 mg once daily is recommended for patients taking concomitant moderate CYP3A inhibitors, such as fluconazole and erythromycin.

Moderate CYP3A inhibitors include, but are not limited to: (1) antifungal medications such as fluconazole (e.g., Diflucan®); or (2) antibiotics such as erythromycin (e.g., Ery-Tab®).

Co-administration of KALYDECO™ with grapefruit juice, which contains one or more components that moderately inhibit CYP3A, may increase exposure of ivacaftor. Therefore, food containing grapefruit or Seville oranges should be avoided during treatment with KALYDECO™ [see Clinical Pharmacology (subsection 12.3 of section V)].

7.2. Inducers of CYP3A

Co-administration with rifampin, a strong CYP3A inducer, significantly decreased ivacaftor exposure (AUC) by approximately 9-fold. Therefore, co-administration with strong CYP3A inducers, such as rifampin, rifabutin, phenobarbital, carbamazepine, phenyloin, and St. John's Wort is not recommended [see Warnings and Precautions (subsection 5.2 of section V) and Clinical Pharmacology (subsection 12.3 of section V)].

It is not known if KALYDECO™ is safe and effective in children under 6 years of age.

KALYDECO™ should not be taken with certain medicines or herbal supplements such as: the antibiotics rifampin (Rifamate®, Rifater®) or rifabutin (Mycobutin®); seizure medications such as phenobarbital, carbamazepine (Tegretol®, Carbatrol®, Equetro®) or phenyloin (Dilantin®, Phenylek®); or St. John's Wort

Potential for Ivacaftor to Affect Other Drugs

7.3. CYP3A and/or P-gp Substrates

Ivacaftor and its M1 metabolite have the potential to inhibit CYP3A and P-gp. Co-administration with midazolam, a sensitive CYP3A substrate, increased midazolam exposure 1.5-fold, consistent with weak inhibition of CYP3A by ivacaftor. Administration of KALYDECO™ may increase systemic exposure of drugs which are substrates of CYP3A and/or P-gp, which may increase or prolong their therapeutic effect and adverse events. Therefore, caution is recommended when co-administering KALYDECO™ with CYP3A and/or P-gp substrates, such as digoxin, cyclosporine, and tacrolimus [see Clinical Pharmacology (subsection 12.3 of section V)].

8. Use in Specific Populations

8.1. Pregnancy

Teratogenic Effects: Pregnancy Category B.

There are no adequate and well-controlled studies of KALYDECO™ in pregnant women. Ivacaftor was not teratogenic in rats at approximately 6 times the maximum recommended human dose (MRHD) (based on summed AUCs for ivacaftor and its metabolites at a maternal dose of 200 mg/kg/day). Ivacaftor was not teratogenic in rabbits at approximately 12 times the MRHD (on an ivacaftor AUC basis at a maternal dose of 100 mg/kg/day, respectively). Placental transfer of ivacaftor was observed in pregnant rats and rabbits. Because animal reproduction studies are not always predictive of human response, KALYDECO™ should be used during pregnancy only if clearly needed.

8.3 Nursing Mothers

Ivacaftor is excreted into the milk of lactating female rats. Excretion of ivacaftor into human milk is probable. There are no human studies that have investigated the effects of ivacaftor on breast-fed infants. Caution should be exercised when KALYDECO™ is administered to a nursing woman.

8.4 Pediatric Use

The safety and efficacy of KALYDECO™ in patients 6 to 17 years of age with CF who have a G551D mutation in the CFTR gene has been demonstrated in 2 placebo-controlled clinical trials. Trial 1 evaluated 161 patients with CF who were 12 years of age or older and Trial 2 evaluated 52 patients with CF who were 6 to 11 years of age [see Clinical Studies (subsection 14.1 of section V)].

The safety and efficacy of KALYDECO™ in patients with CF younger than age 6 years have not been established.

8.5 Geriatric Use

CF is largely a disease of children and young adults. Clinical trials of KALYDECO™ did not include sufficient numbers of patients 65 years of age and over to determine whether they respond differently from younger patients.

8.6 Hepatic Impairment

No dose adjustment is necessary for patients with mild hepatic impairment (Child-Pugh Class A). A reduced dose of 150 mg once daily is recommended in patients with moderate hepatic impairment (Child-Pugh Class B). Studies have not been conducted in patients with severe hepatic impairment (Child-Pugh Class C) but exposure is expected to be higher than in patients with moderate hepatic impairment. Therefore, use with caution at a dose of 150 mg once daily or less frequently in patients with severe hepatic impairment after weighing the risks and benefit of treatment [see Pharmacokinetics (subsection 12.3 of section V)].

8.7 Renal Impairment

KALYDECO™ has not been studied in patients with mild, moderate, or severe renal impairment or in patients with end stage renal disease. No dose adjustment is necessary for patients with mild to moderate renal impairment; however, caution is recommended while using KALYDECO™ in patients with severe renal impairment (creatinine clearance less than or equal to 30 mL/min) or end stage renal disease.

8.8 Patients with CF who are Homozygous for the F508del Mutation in the CFTR Gene

Efficacy results from a double-blind, placebo-controlled trial in patients with CF who are homozygous for the F508del mutation in the CFTR gene showed no statistically significant difference in forced expiratory volume exhaled in one second (FEV1) over 16 weeks of KALYDECO™ treatment compared to placebo [see Clinical Studies (subsection 14.2 of section V)]. Therefore, KALYDECO™ should not be used in patients homozygous for the F508del mutation in the CFTR gene.

10. Overdosage

There have been no reports of overdose with KALYDECO™

The highest single dose used in a clinical study was 800 mg in a solution formulation without any treatment-related adverse events.

The highest repeated dose was 450 mg (in a tablet formulation) every 12 hours for 4.5 days (9 doses) in a trial evaluating the effect of KALYDECO™ on ECGs in healthy subjects. Adverse events reported at a higher incidence compared to placebo included dizziness and diarrhea.

No specific antidote is available for overdose with KALYDECO™. Treatment of overdose with KALYDECO™ consists of general supportive measures including monitoring of vital signs and observation of the clinical status of the patient.

11. Description

The active ingredient in KALYDECO™ tablets is ivacaftor which has the following chemical name: N-(2,4-di-tert-butyl-5-hydroxyphenyl)-1,4-dihydro-4-oxoquinoline-3-carboxamide. Its molecular formula is C₂₄H₂₈N₂O₃ and its molecular weight is 392.49. Ivacaftor has the following structural formula:

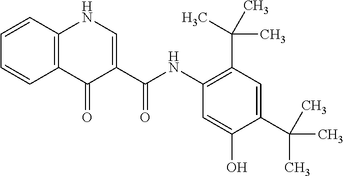 [SMILES: CC(C)(C)c1cc(C(C)(C)C)c(NC(=O)c2c[nH]c3ccccc3c2=O)cc1O]

Ivacaftor is a white to off-white powder that is practically insoluble in water (<0.05 microgram/mL).

KALYDECO™ is available as a light blue capsule-shaped, film-coated tablet for oral administration containing 150 mg of ivacaftor. Each tablet contains the inactive ingredients colloidal silicon dioxide, croscarmellose sodium, hypromellose acetate succinate, lactose monohydrate, magnesium stearate, microcrystalline cellulose, and sodium lauryl sulfate. The tablet film coat contains carnauba wax, FD&C Blue #2, PEG 3350, polyvinyl alcohol, talc, and titanium dioxide. The printing ink contains ammonium hydroxide, iron oxide black, propylene glycol, and shellac.

12. Clinical Pharmacology

12.1 Mechanism of Action

Ivacaftor is a potentiator of the CFTR protein. The CFTR protein is a chloride channel present at the surface of epithelial cells in multiple organs. Ivacaftor facilitates increased chloride transport by potentiating the channel-open probability (or gating) of the G551D-CFTR protein.

In vitro, ivacaftor increased CFTR-mediated transepithelial current (IT) in rodent cells expressing G551D-CFTR protein following addition of a cyclic adenosine monophosphate (cAMP) agonist with an EC₅₀ of 100±47 nM; however, ivacaftor did not increase IT in the absence of cAMP agonist. Ivacaftor also increased IT in human bronchial epithelial cells expressing G551D-CFTR protein following addition of a cAMP agonist by 10-fold with an EC₅₀ of 236±200 nM. Ivacaftor increased the open probability of G551D-CFTR protein in single channel patch clamp experiments using membrane patches from rodent cells expressing G551D-CFTR protein by 6-fold versus untreated cells after addition of PICA and ATP.

12.2 Pharmacodynamics

Sweat Chloride Evaluation

In clinical trials in patients with the G551D mutation in the CFTR gene, KALYDECO™ led to statistically significant reductions in sweat chloride concentration. In two randomized, double-blind, placebo-controlled clinical trials (one in patients 12 and older and the other in patients 6-11 years of age), the mean change in sweat chloride from baseline through week 24 was −48 mmol/L (95% CI −51, −45) and −54 mmol/L (95% CI −62, −47) respectively. These changes persisted through 48 weeks. There was no direct correlation between decrease in sweat chloride levels and improvement in lung function (FEV1).

ECG Evaluation

The effect of multiple doses of ivacaftor 150 mg and 450 mg twice daily on QTc interval was evaluated in a randomized, placebo- and active-controlled (moxifloxacin 400 mg) four-period crossover thorough QT study in 72 healthy subjects. In a study with demonstrated ability to detect small effects, the upper bound of the one-sided 95% confidence interval for the largest placebo adjusted, baseline-corrected QTc based on Fridericia's correction method (QTcF) was below 10 ms, the threshold for regulatory concern.

12.3 Pharmacokinetics

The pharmacokinetics of ivacaftor is similar between healthy adult volunteers and patients with CF.

After oral administration of a single 150 mg dose to healthy volunteers in a fed state, peak plasma concentrations (Tₘₐₓ) occurred at approximately 4 hours, and the mean (±SD) for AUC and Cₘₐₓ were 10600 (5260) ng*hr/mL and 768 (233) ng/mL, respectively.

After every 12 hour dosing, steady-state plasma concentrations of ivacaftor were reached by days 3 to 5, with an accumulation ratio ranging from 2.2 to 2.9.

Absorption

The exposure of ivacaftor increased approximately 2- to 4-fold when given with food containing fat. Therefore, KALYDECO™ should be administered with fat-containing food. Examples of fat-containing foods include eggs, butter, peanut butter, and cheese pizza. The median (range) tₘₐₓ is approximately 4.0 (3.0; 6.0) hours in the fed state.

Distribution

Ivacaftor is approximately 99% bound to plasma proteins, primarily to alpha 1-acid glycoprotein and albumin. Ivacaftor does not bind to human red blood cells.

The mean apparent volume of distribution (Vz/F) of ivacaftor after a single dose of 275 mg of KALYDECO™ in the fed state was similar for healthy subjects and patients with CF. After oral administration of 150 mg every 12 hours for 7 days to healthy volunteers in a fed state, the mean (±SD) for apparent volume of distribution was 353 (122) L.

Metabolism

Ivacaftor is extensively metabolized in humans. In vitro and clinical studies indicate that ivacaftor is primarily metabolized by CYP3A. M1 and M6 are the two major metabolites of ivacaftor in humans. M1 has approximately one-sixth the potency of ivacaftor and is considered pharmacologically active. M6 has less than one-fiftieth the potency of ivacaftor and is not considered pharmacologically active.

Elimination

Following oral administration, the majority of ivacaftor (87.8%) is eliminated in the feces after metabolic conversion. The major metabolites M1 and M6 accounted for approximately 65% of the total dose eliminated with 22% as M1 and 43% as M6. There was negligible urinary excretion of ivacaftor as unchanged parent. The apparent terminal half-life was approximately 12 hours following a single dose. The mean apparent clearance (CL/F) of ivacaftor was similar for healthy subjects and patients with CF. The CL/F (SD) for the 150 mg dose was 17.3 (8.4) L/hr in healthy subjects.

Special Populations

Hepatic Impairment

Patients with moderately impaired hepatic function (Child-Pugh Class B, score 7 to 9) had similar ivacaftor Cₘₐₓ but an approximately two-fold increase in ivacaftor AUC_{0-∞} compared with healthy subjects matched for demographics. Therefore, a reduced KALYDECO™ dose of 150 mg once daily is recommended for patients with moderate hepatic impairment. The impact of mild hepatic impairment (Child-Pugh Class A) on pharmacokinetics of ivacaftor has not been studied, but the increase in ivacaftor AUC_{0-∞} is expected to be less than two-fold. Therefore, no dose adjustment is necessary for patients with mild hepatic impairment. The impact of severe hepatic impairment (Child-Pugh Class C, score 10-15) on pharmacokinetics of ivacaftor has not been studied. The magnitude of increase in exposure in these patients is unknown but is expected to be substantially higher than that observed in patients with moderate hepatic impairment. When benefits are expected to outweigh the risks, KALYDECO™ should be used with caution in patients with severe hepatic impairment at a dose of 150 mg given once daily or less frequently.

Renal Impairment

KALYDECO™ has not been studied in patients with mild, moderate or severe renal impairment (creatinine clearance less than or equal to 30 mL/min) or in patients with end stage renal disease. No dose adjustments are recommended for mild and moderate renal impairment patients because of minimal elimination of ivacaftor and its metabolites in urine (only 6.6% of total radioactivity was recovered in the urine in a human PK study); however, caution is recommended when administering KALYDECO™ to patients with severe renal impairment or end stage renal disease.

Gender

The effect of gender on KALYDECO™ pharmacokinetics was evaluated using population pharmacokinetics of data from clinical studies of KALYDECO™. No dose adjustments are necessary based on gender.

Drug Interactions

Drug interaction studies were performed with KALYDECO™ and other drugs likely to be co-administered or drugs commonly used as probes for pharmacokinetic interaction studies [see Drug Interactions (subsection 7 of section V)].

Dosing recommendations based on clinical studies or potential drug interactions with KALYDECO™ are presented below.

Potential for Ivacaftor to Affect Other Drugs

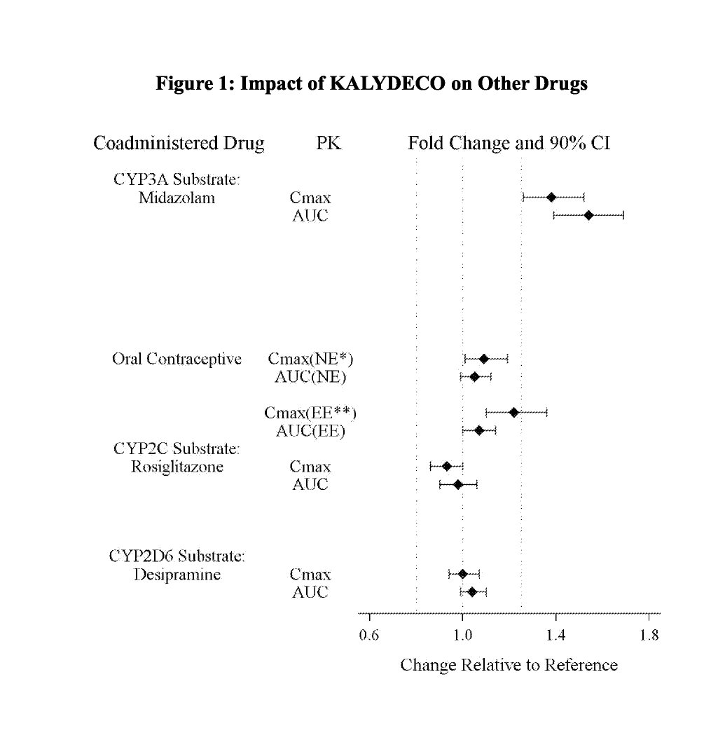

FIG. 1 shows the impact of KALYDECO™ on other drugs. The data obtained with substrates but without co-administration of KALYDECO™ are used as reference. The oral contraceptives used include Norethindrone (NE) and Ethinyl Estradiol (EE). In FIG. 1, “*NE” refers to Norethindrone; “**EE” refers to Ethinyl Estradiol. The vertical lines in FIG. 1 are at 0.8, 1.0 and 1.25, respectively. Dosing recommendations in light of FIG. 1 for co-administered drugs following administration with KALYDECO™ are shown in Table 5 below.

TABLE 5

| Coadministered Drug | Recommendation |
|---|---|
| CYP3A Substrate: | Use with caution and monitor for benzodiazepine- |
| Midazolam | related side effects when using midazolam, |
| | alprazolam, diazepam, triazolam. Appropriate |
| | monitoring is also recommended for other |
| | CYP3A and/or P-gp substrates such as digoxin, |
| | cyclosporine, tacrolimus. |
| Oral Contraceptive | No oral contraceptive dose adjustment |
| CYP2C Substrate: | No dose adjustment for CYP2C8 substrate |
| Rosiglitazone | rosiglitazone. For CYP2C9 substrates, monitoring |
| | is recommended, such as INR with warfarin. |
| CYP2D6 Substrate: | No dose adjustment for CYP2D6 substrate |
| Desipramine | desipramine. |

Potential for Other Drugs to Affect Ivacaftor

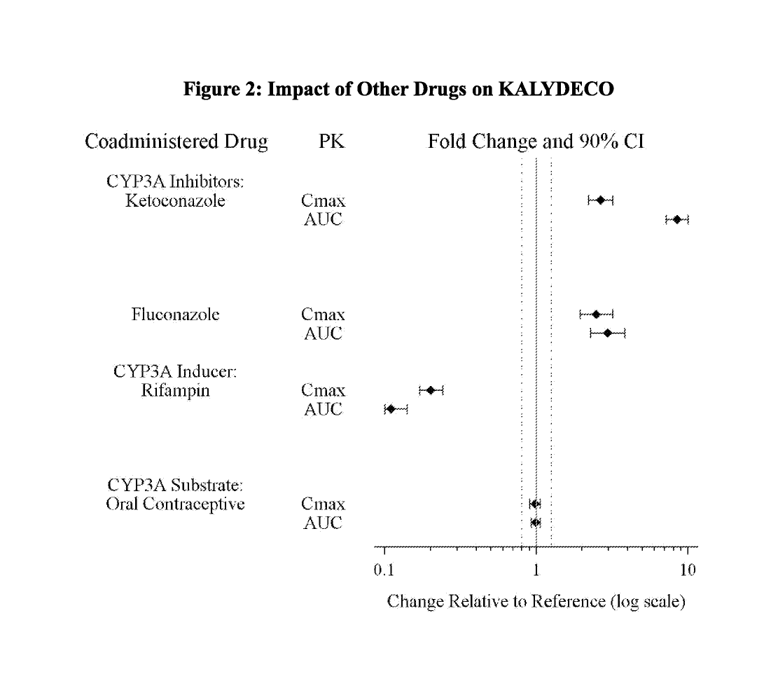

In vitro studies showed that ivacaftor and metabolite M1 were substrates of CYP3A enzymes (i.e., CYP3A4 and CYP3A5). FIG. 2 shows the impact of other drugs on KALYDECO™. The data obtained for KALYDECO™ without co-administration of inducers or inhibitors are used as reference. The vertical lines are at 0.8, 1.0 and 1.25, respectively. Dosing recommendations in light of FIG. 2 for co-administration with CYP3A inhibitors or inducers are shown in Table 6 below.

TABLE 6

| Coadministered Drug | Recommendations |
|---|---|
| CYP3A Inhibitors: | 150 mg KALYDECO™ twice-a-week when used |
| Ketoconazole | with strong inhibitors such as ketoconazole, |
| | itraconazole, posaconazole, voriconazole, |
| | clarithromycin and telithromycin. |
| Fluconazole | 150 mg KALYDECO™ once-daily for moderate |
| | inhibitors such as fluconazole and erythromycin |
| CYP3A Inducer: | Concomitant use with strong CYP3A inducers |
| Rifampin | such as rifampin, rifabutin, phenobarbital, |
| | phenytoin, carbamazepine and St. John's wort is |
| | not recommended |
| CYP3A Substrate: | No KALYDECO™ dose adjustment |
| Oral contraceptive | |

13. Nonclinical Toxicology

13.1 Carcinogenesis, Mutagenesis, and Impairment of Fertility

Two-year studies were conducted in mice and rats to assess carcinogenic potential of KALYDECO™. No evidence of tumorigenicity was observed in mice or rats at ivacaftor oral doses up to 200 mg/kg/day and 50 mg/kg/day, respectively (approximately equivalent to and 3 to 5 times the MRHD, respectively, based on summed AUCs of ivacaftor and its metabolites).

Ivacaftor was negative for genotoxicity in the following assays: Ames test for bacterial gene mutation, in vitro chromosomal aberration assay in Chinese hamster ovary cells, and in vivo mouse micronucleus test.

Ivacaftor impaired fertility and reproductive performance indices in male and female rats at 200 mg/kg/day (approximately 5 and 6 times, respectively, the MRHD based on summed AUCs of ivacaftor and its metabolites). Increases in prolonged diestrus were observed in females at 200 mg/kg/day. Ivacaftor also increased the number of females with all nonviable embryos and decreased corpora lutea, implantations, and viable embryos in rats at 200 mg/kg/day (approximately 6 times the MRHD based on summed AUCs of ivacaftor and its metabolites) when dams were dosed prior to and during early pregnancy. These impairments of fertility and reproductive performance in male and female rats at 200 mg/kg/day were attributed to severe toxicity. No effects on male or female fertility and reproductive performance indices were observed at ≦100 mg/kg/day (approximately 3 times the MRHD based on summed AUCs of ivacaftor and its metabolites).

13.2 Animal Toxicology and/or Pharmacology

Cataracts were seen in juvenile rats dosed with ivacaftor from postnatal day 7-35 at dose levels of 10 mg/kg/day and higher (approximately 0.12 times the MRHD based on summed AUCs of ivacaftor and its metabolites). This finding has not been observed in older animals.

14. Clinical Studies

14.1 Trials in Patients with CF Who have a G551D Mutation in the CFTR Gene

Dose Ranging:

Dose ranging for the clinical program consisted primarily of one double-blind, placebo-controlled, cross-over trial in 39 adult (mean age 31 years) Caucasian patients with CF who had FEV1≧40% predicted. Twenty patients with median predicted FEV1 at baseline of 56% (range: 42% to 109%) received KALYDECO™ 25, 75, 150 mg or placebo every 12 hours for 14 days and 19 patients with median predicted FEV1 at baseline of 69% (range: 40% to 122%) received KALYDECO™ 150, 250 mg or placebo every 12 hours for 28 days. The selection of the 150 mg every 12 hours dose was primarily based on nominal improvements in lung function (pre-dose FEV1) and changes in pharmacodynamic parameters (sweat chloride and nasal potential difference). The twice-daily dosing regimen was primarily based on an apparent terminal plasma half-life of approximately 12 hours. Selection of the 150 mg dose of KALYDECO™ for children 6 to 11 years of age was based on achievement of comparable pharmacokinetics as those observed for adult patients.

Efficacy:

The efficacy of KALYDECO™ in patients with CF who have a G551D mutation in the CFTR gene was evaluated in two randomized, double-blind, placebo-controlled clinical trials in 213 clinically stable patients with CF (109 receiving KALYDECO™ 150 mg twice daily). All eligible patients from these trials were rolled over into an open-label extension study.

Trial 1 evaluated 161 patients with CF who were 12 years of age or older (mean age 26 years) with baseline FEV1 between 40-90% predicted [mean FEV1 64% predicted (range: 32% to 98%)]. Trial 2 evaluated 52 patients who were 6 to 11 years of age (mean age 9 years) with baseline FEV1 between 40-105% predicted [mean FEV1 84% predicted (range: 44% to 134%)]. Patients who had persistent Burkholderia cenocepacia, dolosa, or Mycobacterium abcessus isolated from sputum at screening and those with abnormal liver function defined as 3 or more liver function tests (ALT, AST, AP, GGT, total bilirubin)≧3 times the upper limit of normal were excluded.

Patients in both trials were randomized 1:1 to receive either 150 mg of KALYDECO™ or placebo every 12 hours with food containing fat for 48 weeks in addition to their prescribed CF therapies (e.g., tobramycin, dornase alfa). The use of inhaled hypertonic saline was not permitted.

The primary efficacy endpoint in both studies was improvement in lung function as determined by the mean absolute change from baseline in percent predicted pre-dose FEV1 through 24 weeks of treatment.

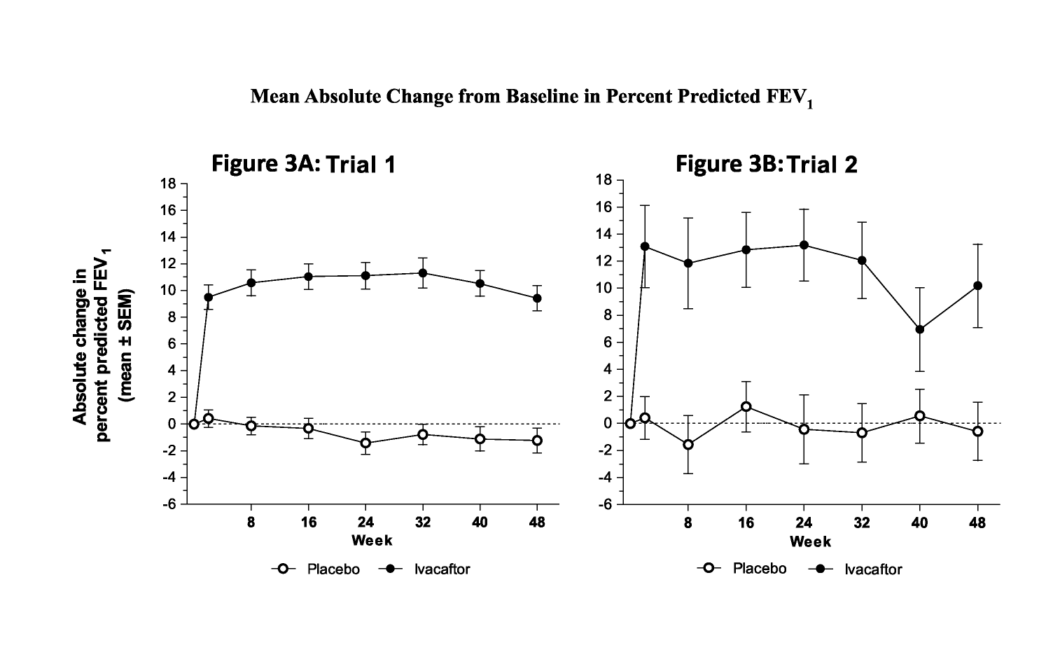

In both studies, treatment with KALYDECO™ resulted in a significant improvement in FEV1. The treatment difference between KALYDECO™ and placebo for the mean absolute change in percent predicted FEV1 from baseline through Week 24 was 10.6 percentage points (P<0.0001) in Trial 1 and 12.5 percentage points (P<0.0001) in Trial 2 (FIGS. 3A and 3B). These changes persisted through 48 weeks. Improvements in percent predicted FEV1 were observed regardless of age, disease severity, sex, and geographic region. The primary endpoint in FIGS. 3A and 3B was assessed at the 24-week time point.

Other efficacy variables included absolute change in sweat chloride from baseline to week 24 [discussed in Clinical Pharmacology (12.2)], time to first pulmonary exacerbation through week 48 (Trial 1 only), absolute change in weight from baseline to week 48, and improvement in cystic fibrosis symptoms including relevant respiratory symptoms such as cough, sputum production, and difficulty breathing. For the purpose of the study, a pulmonary exacerbation was defined as a change in antibiotic therapy (IV, inhaled, or oral) as a result of 4 or more of 12 pre-specified sino-pulmonary signs/symptoms. Patients treated with KALYDECO™ demonstrated statistically significant improvements in risk of pulmonary exacerbations, CF symptoms (in Trial 1 only), and gain in body weight (Table 7). Weight data, when expressed as body mass index normalized for age and sex in patients <20 years of age, was consistent with absolute change from baseline in weight.

TABLE 7
Effect of KALYDECO™ on Other Efficacy
Endpoints in Trials 1 and 2

| | Trial 1 | | Trial 2 | |
|---|---|---|---|---|
| | Treatment | | Treatment | |
| | difference^{a} | | difference^{a} | |
| Endpoint | (95% CI) | P value | (95% CI) | P value |

Mean absolute change from baseline in
CF symptom score (points)

| | | | | |
|---|---|---|---|---|
| Through Week 24 | 8.1 | <0.0001 | 6.1 | 0.1092 |
| | (4.7, 11.4) | | (−1.4, 13.5) | |
| Through Week 48 | 8.6 | <0.0001 | 5.1 | 0.1354 |
| | (5.3, 11.9) | | (−1.6, 11.8) | |

Relative risk of pulmonary exacerbation

| | | | | |
|---|---|---|---|---|
| Through Week 24 | 0.40^{b} | 0.0016 | NA | NA |
| Through Week 48 | 0.46^{b} | 0.0012 | NA | NA |

Mean absolute change from baseline in body weight (kg)

| | | | | |
|---|---|---|---|---|
| At Week 24 | 2.8 | <0.0001 | 1.9 | 0.0004 |
| | (1.8, 3.7) | | (0.9, 2.9) | |
| At Week 48 | 2.7 | 0.0001 | 2.8 | 0.0002 |
| | (1.3, 4.1) | | (1.3, 4.2) | |

CI: confidence interval;
NA: not analyzed due to low incidence of events
^{a}Treatment difference = effect of KALYDECO™ - effect of Placebo
^{b}Hazard ratio for time to first pulmonary exacerbation

14.2 Trial in Patients Homozygous for the F508del Mutation in the CFTR Gene

Trial 3 was a 16-week randomized, double-blind, placebo-controlled, parallel-group trial in 140 patients with CF age 12 years and older who were homozygous for the F508del mutation in the CFTR gene and who had FEV₁≧40% predicted. Patients were randomized 4:1 to receive KALYDECO™ 150 mg (n=112) every twelve hours or placebo (n=28) in addition to their prescribed CF therapies. The mean age of patients enrolled was 23 years and the mean baseline FEV₁ was 79% predicted (range 40% to 129%). As in Trials 1 and 2, patients who had persistent Burkholderia cenocepacia, dolosa, or Mycobacterium abcessus isolated from sputum at screening and those with abnormal liver function defined as 3 or more liver function tests (ALT, AST, AP, GGT, total bilirubin)≧3 times the upper limit of normal were excluded. The use of inhaled hypertonic saline was not permitted.

The primary endpoint was improvement in lung function as determined by the mean absolute change from baseline through Week 16 in percent predicted FEV1. Treatment with KALYDECO™ resulted in no improvement in FEV1 relative to placebo in patients with CF homozygous for the F508del mutation in the CFTR gene [mean absolute change from baseline through Week 16 in percent predicted FEV1 was 1.5% and −0.2% for patients in the KALYDECO™ and placebo-treated groups, respectively (p=0.15)]. There were no meaningful differences between patients treated with KALYDECO™ compared to placebo for secondary endpoints (change in CF symptoms, change in weight, or change in sweat chloride concentration).

16. How Supplied/Storage and Handling

KALYDECO™ (ivacaftor) is supplied as light blue, film-coated, capsule-shaped tablets containing 150 mg of ivacaftor. Each tablet is printed with the characters “V 150” on one side and plain on the other, and is packaged as follows:

56-count carton (contains 4 individual blister cards of 14 tablets per card) NDC 51167-200-01
60-count bottle NDC 51167-200-02

Store at 20-25° C. (68-77° F.); excursions permitted to 15-30° C. (59-86° F.) [see USP Controlled Room Temperature].

17. Patient Counseling Information

17.1 Transaminase (ALT or AST) Elevations and Monitoring

Inform patients that elevation in liver tests have occurred in patients treated with KALYDECO™. Liver function tests will be performed prior to initiating KALYDECO™, every 3 months during the first year of treatment and annually thereafter [see Warnings and Precautions (subsection 5.1 of section V)].

17.2 Drug Interactions with CYP3A Inducers and Inhibitors

Ask patients to tell you all the medications they are taking including any herbal supplements or vitamins. Co-administration of KALYDECO™ with strong CYP3A inducers (e.g., rifampin, St. John's Wort) is not recommended as they may reduce the therapeutic effectiveness of KALYDECO™. Reduction of the dose of KALYDECO™ to 150 mg twice-a-week is recommended when co-administered with strong CYP3A inhibitors, such as ketoconazole. Dose reduction to 150 mg once daily is recommended when co-administered with moderate CYP3A inhibitors, such as fluconazole. Food containing grapefruit or Seville oranges should be avoided [see Drug Interactions (subsections 7.1, 7.2 of section V) and Clinical Pharmacology (subsection 12.3 of section V)].

17.3 Use in Patients with Hepatic Impairment

Inquire and/or assess whether patients have liver impairment. Reduce the dose of KALYDECO™ in patients with moderately impaired hepatic function (Child-Pugh Class B, score 7 to 9) to 150 mg once daily. KALYDECO™ has not been studied in patients with severe hepatic impairment (Child-Pugh Class C, score 10-15); however, exposure is expected to be substantially higher than that observed in patients with moderate hepatic impairment. When benefits are expected to outweigh the risks, KALYDECO™ should be used with caution in patients with severe hepatic impairment at a dose of 150 mg given once daily or less frequently. No dose adjustment is recommended for patients with mild hepatic impairment (Child-Pugh Class A, score 5-6) [see Clinical Pharmacology (subsection 12.3 of section V)]

17.4 Take with Fat-Containing Food

Inform your patients that KALYDECO™ is best absorbed by the body when taken with fatty food. A typical CF diet will satisfy this requirement. Examples include eggs, butter, peanut butter, cheese pizza, etc.

### VI. PK/PD Modeling Guided Ivacaftor Dose Rationale

Pharmacokinetic/Pharmacodynamic Relationships

Based on pooled data from Phase 2a and Phase 3 studies in patients with a G551D mutation, population PK/PD analysis showed a relationship between FEV₁ and ivacaftor exposure in an Eₘₐₓ model with an EC₅₀ of 45 ng/mL and a corresponding EC₉₀ of 405 ng/mL. Therefore, median Cₘᵢₙ at EC₉₀ was chosen as the target PK parameter for efficacy.

### VII. Simulations Guided Ivacaftor Dose Adjustment

Hepatic Impairment

Following a single dose of 150 mg of ivacaftor, subjects with moderately impaired hepatic function (Child-Pugh Class B, score 7 to 9) had similar ivacaftor Cₘₐₓ (mean (±SD) of 735 (331) ng/mL), but an approximately two-fold increase in ivacaftor AUC_{0-∞} (mean (±SD) of 16800 (6140) ng*hr/mL) compared with healthy subjects matched for demographics. Simulations for predicting the steady-state exposure of ivacaftor showed that by reducing the dosage from 150 mg q12h to 150 mg once daily, subjects with moderate hepatic impairment would have comparable steady-state Cₘᵢₙ values as those obtained with a dose of 150 mg q12h in subjects with CF. Therefore, a reduced dose of 150 mg once daily is recommended in patients with moderate hepatic impairment.

### VIII. Simulations Guided Ivacaftor Dose Adjustment

CYP3A Inhibitors

Ivacaftor is a sensitive CYP3A substrate. Co-administration with ketoconazole, a strong CYP3A inhibitor, increased ivacaftor exposure [measured as area under the curve (AUC)] by 8.5-fold and hydroxymethyl-ivacaftor (M1) exposure by 1.7-fold. A reduction of the ivacaftor dose to 150 twice-a-week is recommended for co-administration with strong CYP3A inhibitors, such as ketoconazole, itraconazole, posaconazole, voriconazole, telithromycin, and clarithromycin.

Co-administration with fluconazole, a moderate inhibitor of CYP3A, increased ivacaftor exposure by 3-fold and M1 exposure by 1.9-fold. A reduction of the ivacaftor dose to 150 mg once daily is recommended for patients taking concomitant moderate CYP3A inhibitors, such as fluconazole and erythromycin.

### Other Embodiments

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling. Furthermore, the foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims.

What is claimed is:

1-34. (canceled)

35. A product comprising:
a) ivacaftor formulated as KALYDECO™ or bioequivalent drug product thereof; and
b) prescribing information for administering KALYDECO™ or a bioequivalent drug product thereof, wherein the prescribing information includes:
i) dosage and administration information for adults and pediatric patients 6 years and older instructing the administration of one 150 mg tablet of KALYDECO™ or a bioequivalent drug product thereof taken orally every 12 hours with fat-containing food;
ii) dosage and administration information to reduce the dose of KALYDECO™ or a bioequivalent drug product thereof in patients with moderate or severe hepatic impairment; and
iii) dosage and administration information to reduce the dose of KALYDECO™ or a bioequivalent drug product thereof when co-administered with drugs that are moderate or strong CYP3A inhibitors.

36. The product of claim 35, wherein the prescribing information describes fat-containing food as selected from eggs, butter, peanut butter, and cheese pizza.

37. The product of claim 35, wherein the prescribing information recommends a reduced dose of 150 mg of KALYDECO™ or a bioequivalent drug product thereof once daily in patients with moderate hepatic impairment.

38. The product of claim 35, wherein the prescribing information recommends a reduced dose of 150 mg of KALYDECO™ or a bioequivalent drug product thereof once daily or less frequently in patients with severe hepatic impairment.

39. The product of claim 35, wherein the prescribing information recommends reducing the dose of KALYDECO™ or a bioequivalent drug product thereof to 150 mg twice-a-week when co-administered with strong CYP3A inhibitors.

40. The product of claim 39, wherein the prescribing information describes strong CYP3A inhibitors as selected from ketoconazole, itraconazole, posaconazole, voriconazole, telithromycin, and clarithmycin.

41. The product of claim 35, wherein the prescribing information recommends reducing the dose of KALYDECO™ or a bioequivalent drug product thereof to 150 mg once daily when co-administered with moderate CYP3A inhibitors.

42. The product of claim 41, wherein the prescribing information describes moderate CYP3A inhibitors as fluconazole or erthromycin.

43. A product comprising:
a) ivacaftor formulated as KALYDECO™ or bioequivalent drug product thereof; and
b) prescribing information for administering KALYDECO™ or a bioequivalent drug product thereof, wherein the prescribing information includes:
i) drug interaction information to reduce the dose of KALYDECO™ or a bioequivalent drug product thereof to 150 mg of ivacaftor twice-a-week when co-administered with a strong CYP3A inhibitors;
ii) drug interaction information to reduce the dose of KALYDECO™ or a bioequivalent drug product thereof to 150 mg of ivacaftor once daily when co-administered with a moderate CYP3A inhibitors; and
iii) drug interaction information to avoid food containing grapefruit or Seville oranges.

44. A product comprising:
a) ivacaftor formulated as KALYDECO™ or bioequivalent drug product thereof; and
b) prescribing information for administering KALYDECO™ or a bioequivalent drug product thereof, wherein the prescribing information includes:
i) warnings and precautions regarding elevated transaminases ALT or AST, wherein the prescribing information advises that transaminases ALT and AST should be assessed prior to initiating KALYDECO™ or a bioequivalent drug product thereof, every 3 months during the first year of treatment of KALYDECO™ or a bioequivalent drug product thereof, and annually thereafter;
ii) warnings and precautions regarding elevated transaminases ALT or AST, wherein the prescribing information advises that dosing of KALYDECO™ or a bioequivalent drug product thereof should be interrupted in patients with ALT or AST of greater than 5 times the upper limit of normal; and
iii) warnings and precautions regarding CYP3A inducers, wherein the prescribing information advises that
a) concomitant use of KALYDECO™ or a bioequivalent drug product thereof with strong CYP3A inducers substantially decreases exposure of ivacaftor which may diminish effectiveness, and
b) co-administration is not recommended.

45. The product of claim 44, wherein the prescribing information describes CYP3A inducers as selected from rifampin, rifabutin, phenobarbital, carbamazepine, phenyloin, and St. John's Wort.

46. A product comprising:
a) ivacaftor formulated as KALYDECO™ or bioequivalent drug product thereof; and
b) prescribing information for administering KALYDECO™ or a bioequivalent drug product thereof, wherein the package insert includes:
i) information regarding the potential for ivacaftor to affect other drugs including CYP3A and P-gp substrates, wherein the prescribing information advises caution when co-administering KALYDECO™ or bioequivalent drug product thereof with CYP3A and/or P-gp substrates.

47. The product of claim 46, wherein the prescribing information describes CYP3A and/or P-gp substrates as selected from midazolam, alprazolam, diazepam, triaolam, digoxin, cyclosporine, and tacrolimus.

48. The product of any one of claims 35 to 47, wherein the prescribing information is provided as a package insert.
